(19) United States
(12) Patent Application Publication
(10) Pub. No.: US 2024/0229146 A9
(43) Pub. Date: Jul. 11, 2024

(54) DIAGNOSIS AND RISK DETERMINATION FOR HEAD AND NECK CANCER

(71) Applicants: ONCGNOSTICS GMBH, Jena (DE); UNIVERSITÄTSKLINIKUM JENA, Jena (DE)

(72) Inventors: Orlando GUNTINAS-LICHIUS, Jena (DE); Daniel STEINBACH, Jena (DE); Martina SCHMITZ, Jena (DE); Alfred HANSEL, Jena (DE)

(21) Appl. No.: 18/278,982

(22) PCT Filed: Feb. 26, 2021
(86) PCT No.: PCT/EP2021/054926

§ 371 Date: Aug. 25, 2023

(51) Int. Cl. C12Q 1/6886

(52) U.S. Cl. CPC C12Q 1/6886; C12Q 2600/118; C12Q 2600/154

(57) ABSTRACT

Methods for the determination of the risk for developing a head and neck cancer are described. The method is based on the determination of the methylation status of genomic DNA sequences located between the genes ZNF823 and ZNF833, wherein when the sequences are methylated the risk for developing a head and neck cancer is increased.

### TECHNICAL FIELD OF THE INVENTION

The present invention relates to a method for determining/diagnosing the likelihood of the presence of a cancer of the head and neck in a patient, as well as determining the risk of developing cancer of the head and neck in a patient. In particular, the method involves determining the methylation status of the genomic region between the gene ZNF823 and the gene ZNF833 on chromosome 19 in a biological sample obtained from the patient, wherein when the genomic region is methylated in the sample, the patient has or has an increased likelihood of having a cancer of the head and neck and/or has an increased risk for developing a cancer of the head and neck.

### BACKGROUND OF THE INVENTION

According to the “International Agency for Research on Cancer” (IARC), around 657,000 people worldwide were diagnosed with head and neck cancer (cancer of the oral cavity, lips, naso-, oro-, hypopharynx) in 2015, and around 336,000 people died of such cancer (Bray et al., 2018, Global cancer statistics 2018: GLOBOCAN estimates of incidence and mortality worldwide for 36 cancers in 185 countries, CA Cancer J Clin 68:394-424). The main reason for the relatively high mortality rate with this cancer is the late discovery of the disease due to a lack of early detection tests, and in some cases the poor treatability. A further increase is forecast for the incidence rate of these cancers of the head and neck. The growing number of new cases is probably also related to the increasing spread of infections with human papillomaviruses (HPV), which can cause tumors in the head and neck area.

In addition to alcohol and tobacco abuse, infections with the human papillomavirus are among the main risk factors for the disease. They are mainly related to the development of oropharyngeal carcinoma. The tonsils are most affected. Some tests available for diagnosing these tumors are dependent on the patient's HPV infection status and are based on the detection of the viruses.

Many publications have shown that methylation markers are generally suitable for molecular diagnostics in the field of early detection of the certain cancers, such as cervical carcinoma. For example, Wang et al., 2008, Cancer Res. 68:2489 describe the identification of new methylation markers in cervical carcinoma. Further, Huang et al., 2008, Abstract #50, 99th Annual Meeting of the American Association for Cancer Research, San Diego, CA, USA describe the hypermethylation of CIDEA and RXFP3 as potential epigenetic markers for ovarian cancer.

EP Patent No. 2 478 117 B1 describes detection of hypermethylation of the promoter/5′-regions of the ASTN1 and ZNF671 genes for the detection of CIN3 and cervical carcinomas and Schmitz et al., 2017, Clin. Epigenetics 9: Article number 118, describe the use of DNA methylation markers such as ASTN1, DLX1, ITGA4, RXFP3, SOX17, and ZNF671 for the triage of high-risk papillomavirus DNA-positive women.

There are many diagnostic methods which are useful for determining the likelihood that a patient will develop or has cancer. Nonetheless, there remains a need in the art for methods to determine the risk for developing or diagnosing a head and neck cancer in patients, for example where there is no indication of cancer in the tissue of the head and neck. The invention described below fulfills this need.

### SUMMARY OF THE INVENTION

The present invention is based, at least in part, on the inventors' discovery that the methylation status of a region located between the genes ZNF823 and ZNF833 correlates with the likelihood/diagnosis or the relative risk of having or developing a cancer of the head and neck. This region is located on human chromosome 19, between nucleotide 11,686,870 and nucleotide 11,721,264 in the current full final human genome assembly hg38 from December 2013. This region is depicted in FIG. 1 and the sequence is set forth in SEQ ID NO:1. This region is characterized by a high proportion of the nucleotides cytosine (C) and guanine (G) and contains a CpG island of at least 59 CpG dinucleotides.

Accordingly, in an aspect the present invention is directed to a method for determining the risk for developing a cancer of the head and neck in a human patient, comprising determining the methylation status of the genomic region between the gene ZNF823 and the gene ZNF833 on chromosome 19 in a biological sample obtained from the patient. In an aspect, the present invention is directed to a method for determining the likelihood of the presence of a cancer of the head and neck in a human patient, comprising determining the methylation status of the genomic region between the gene ZNF823 and the gene ZNF833 on chromosome 19 in a biological sample obtained from the patient. In an aspect, the present invention is directed to a method for diagnosing the presence of a cancer of the head and neck in a human patient, comprising determining the methylation status of the genomic region between the gene ZNF823 and the gene ZNF833 on chromosome 19 in a biological sample obtained from the patient. In an embodiment, when the genomic region is methylated in the biological sample, the patient has an increased risk for developing a cancer of the head and neck, or the patient has an increased likelihood that a cancer of the head and neck is present, or the patient has a cancer of the head and neck. In an embodiment, the level of methylation of the genomic region can be 1%, 2%, 3%, 4%, 5%, 10%, 15%, 20%, 30%, 40%, 50%, 60%, 70%, 80%, 90%, 95%, or 100% of the maximum level of methylation.

In an embodiment, the genomic region between the gene ZNF823 and the gene ZNF833 is from nucleotide 11,686,870 to nucleotide 11,721,264 of human chromosome 19. In an embodiment, the genomic region is from nucleotide 11,694,447 to nucleotide 11,695,085 of human chromosome 19. In the present invention, the genes and nucleotide positions are as set forth in the current full final human genome assembly hg38 from December 2013.

In an embodiment, at least one of the CpG dinucleotides located between nucleotide 11,694,447 and nucleotide 11,695,085 of human chromosome 19 can be methylated. In an embodiment, at least 0.5%, 1%, 2%, 3%, 4%, 5%, 10%, 20%, 30%, 40%, 50%, 60%, 70%, 80%, 90%, 95%, or 100% of the CpG dinucleotides located between nucleotide 11,694,447 and nucleotide 11,695,085 of human chromosome 19 can be methylated. The methylation status of the region can be compared to the methylation status of the same region in a control sample. A control sample can be a sample obtained from a tissue in which it is known that the genomic region between the gene ZNF823 and the gene ZNF833 on chromosome 19 is not methylated or can be a standard reflecting a known value or status of methylation. A control sample can also be a biological sample obtained from a different patient, in which it has been determined that the genomic region between the gene ZNF823 and the gene ZNF833 on chromosome 19 is not methylated.

The cancer of the head and neck can be cancer of the mouth and tongue, cancer of the nose, cancer of the nasopharynx, cancer of the throat, cancer of the hypopharynx, cancer of the larynx, cancer of the trachea, cancer of the esophagus, cancer of the tonsils, cancer of the sinuses, or cancer of the salivary glands.

In an embodiment, the biological sample obtained from the patient can contain cells of the head and/or neck. In an embodiment, the biological sample can be a tissue biopsy, preferably that comprises cells of the throat, neck, mouth, nasal passages, esophagus, tonsils, and/or saliva glands. In an embodiment, the biological sample is saliva, blood, sputum, bronchial aspirate, urine, stool, bile, gastrointestinal secretions, or lymph fluid, preferably that comprises cells of the throat, neck, mouth, nasal passages, esophagus, tonsils, and/or saliva glands.

The methods of the present invention can comprise a further step of determining the methylation status of one or more regions of genomic DNA associated with a gene selected from the group consisting of ZNF773, ZNF671, ZIC1, HOXA9, and PAX6 in a different biological sample obtained from the patient or in the same biological sample obtained from the patient. In an embodiment, the region of genomic DNA associated with the gene is the promoter/enhancer region of the gene. In an embodiment, when the one or more regions of genomic DNA associated with a gene selected from the group consisting of ZNF773, ZNF671, ZIC1, HOXA9, and PAX6 is methylated in the biological sample, the patient may have more of an increased risk for developing a cancer of the head and neck, or the patient has more of an increased likelihood that a cancer of the head and neck is present, or the patient has a cancer of the head and neck. In an embodiment, when the one or more regions of genomic DNA associated with a gene selected from the group consisting of ZNF773, ZNF671, ZIC1, HOXA9, and PAX6 is methylated in the biological sample, the determination of the increased risk, increased likelihood or that a cancer of the head and neck is present can be made with a higher degree of (statistical) accuracy.

In an embodiment, the region of genomic DNA associated with ZNF773 can be between nucleotide 57,499,757 and nucleotide 57,500,375 of human chromosome 19. In an embodiment, the region of genomic DNA associated with ZNF671 can be between nucleotide 57,727,218 and nucleotide 57,727,600 of human chromosome 19. In an embodiment, the region of genomic DNA associated with ZIC1 can be between nucleotide 147,412,556 and nucleotide 147,412,790 of human chromosome 3. In an embodiment, the region of genomic DNA associated with HOXA9 can be between nucleotide 27,164,297 and nucleotide 27,166,843 of human chromosome 7. In an embodiment, the region of genomic DNA associated with PAX6 can be between nucleotide 31,798,513 and nucleotide 31,799,868 of human chromosome 11.

The patient can be infected with papillomavirus or can be free of papillomavirus infection.

The determination of the methylation status of one or more of the genomic regions can be performed using any appropriate method known in the art. For example, the methylation status can be determined by nanopore sequencing, or can be determined by methylation-specific PCR (MSP), preferably wherein the MSP is a quantitative MSP (QMSP), and optionally using fluorescent probes. Also, the methylation status can be determined by a methylation-sensitive restriction enzyme, regardless of whether the methylation-sensitive enzyme cleaves methylated DNA or whether the methylation-sensitive enzyme cleaves unmethylated DNA.

In an embodiment, after the patient has been determined to have an increased risk for developing a cancer of the head and neck, or the patient has an increased likelihood that a cancer of the head and neck is present, or the patient has a cancer of the head and neck, the method further comprises administering to the patient a medicament to treat the cancer or to prevent development of the cancer. Any such medicament known in the art suitable for preventing or treating cancer can be administered to the patient having an increased risk for developing a cancer of the head and neck. In an embodiment, the medicament is an anti-inflammatory agent, preferably a non-steroidal anti-inflammatory agent or the medicament is a methylation inhibitor, such as azacytidine or decitabine. In an embodiment, the patient having the increased risk for developing a head and neck cancer can be treated by vaccinating the patient against papillomavirus, e.g., human papillomavirus. In an embodiment, the papillomavirus to be vaccinated against preferably can be a strain of the virus known to cause or contribute to causing cancer, such as HPV strains 16 and 18.

The present invention is also directed to a method for selecting a patient to undergo more frequent screening for developing a cancer of the head and neck, comprising selecting a human patient in which the genomic region between the gene ZNF823 and the gene ZNF833 on chromosome 19 in a biological sample obtained from the patient is methylated. In an embodiment, the more frequent screening can be histopathology-based screening, for example, every 12 months, preferably every 6 months, more preferably every 3 months.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (cf, e.g., Molecular Cloning: A Laboratory Manual, 4th Edition, M. R. Green, J. Sambrook et al. eds., Cold Spring Harbor Laboratory Press, Cold Spring Harbor 2012).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The present invention, inter alia, allows for the identification of human patients who have an increased risk for developing a head and neck cancer. The identification of such patients is due to the fact that in tissues or other biological samples obtained from patients where genomic DNA sequences located between genes ZNF823 and ZNF833 on chromosome 19 are methylated, a significantly increased risk for developing a head and neck cancer exists. Once such patients having the increased risk for developing or having a head and neck cancer have been identified, such patients can be monitored more frequently for the appearance of a head and neck cancer using standard histopathological methods in order to increase the likelihood of early detection and/or can be treated to prevent the development of a head and neck cancer.

In addition to or in combination with the region located between the genes ZNF823 and ZNF833 on chromosome 19, the methylation of one or more regions of genomic DNA associated with a gene selected from the group consisting of ZNF773, ZNF671, ZIC1, HOXA9, and PAX6 can be determined.

In certain embodiments of the invention where the subject is a non-human subject, the genes associated with the sequences for which the methylation status is determined will be the respective homologous genes in the respective non-human subject. In an embodiment relating to non-human subjects, the genomic DNAs are those regions in the non-human chromosome(s) having the strongest homology/identity with the human sequences of the respective genes and/or any portion thereof.

ZNF671 (GenBank accession number NM_024833, contained in NC_000019.10), which is a transcription factor having a typical zinc finger motif. This protein plays an important role in transcription repression and is involved in cell differentiation, proliferation, apoptosis and tumor suppression. The expression of the protein is controlled, among other things, by the methylation status of the promoter region. Studies have shown epigenetic regulation through DNA methylation for various tumor entities (urothelial carcinoma, cervical carcinoma, carcinomas in the head and neck area) (Yeh et al., 2015, Methylomics analysis identifies ZNF671 as an epigenetically repressed novel tumor suppressor and a potential non-invasive biomarker for the detection of urothelial carcinoma, Oncotarget. 6:29555-29572; Tian et al., 2014, Prognostication of patients with clear cell renal cell carcinomas based on quantification of DNA methylation levels of CpG island methylator phenotype marker genes, BMC Cancer 14:772; Hansel et al., 2014, A Promising DNA Methylation Signature for the Triage of High-Risk Human Papillomavirus DNA-Positive Women. PLoS ONE 9(3): e91905). In connection with nasopharyngeal carcinoma, data have already been published that show that aberrant hypermethylation of ZNF671 is related to the progression of the disease (Zhang et al., 2017, Journal of Experimental & Clinical Cancer Research 36:147; Zhang et al., 2017, Liquid Biopsy for Cancer: Circulating Tumor Cells, Circulating Free DNA or Exosomes?, Cellular Physiology and Biochemistry 41:755-768; Zhao et al., 2017, BMC Cancer 17:489).

ZNF773 (GenBank accession number NM_001304334.1, contained in NC_000019.10) has been described to be hypermethylated in tumor tissues of HPV-positive oropharyngeal carcinomas (Ren et al., 2018, Discovery and development of differentially methylated regions in human papillomavirus-related oropharyngeal squamous cell carcinoma, Int. J. Cancer 143:2425-2436).

ZIC1 (GenBank accession number NM_003412.4, contained in NC_000003.12) belongs to the family of the zinc finger proteins of the cerebellum (ZIC). This protein functions as a transcription factor in the neurogenesis of early development. Also, the protein has been described as a potential transcriptional regulator of the gene for apolipoprotein E (lipid metabolism) (Salero et al., 2001, Transcription Factors ZIC1 and ZIC2 Bind and Transactivate the Apolipoprotein E Gene Promoter, J. Biol. Chem. 276:1881-1888). Further, hypermethylation in gastric cancer and head and neck tumors has been described for ZIC1 (Lin et al., 2017, Combined Detection of Plasma ZIC1, HOXD10 and RUNX3 Methylation is a Promising Strategy for Early Detection of Gastric Cancer and Precancerous Lesions, Journal of Cancer 8:1038-1044; Paluszczak et al., 2017, Prognostic significance of the methylation of Wnt pathway antagonists—CXXC4, DACT2, and the inhibitors of sonic hedgehog signaling—ZIC1, ZIC4, and HHIP in head and neck squamous cell carcinomas, Clin. Oral Invest. 21:1777-1788).

HOXA9 (homeobox gene A9) (GenBank accession number NM_152739.4, contained in NC_000007.14) and PAX6 (paired box 6 gene) (GenBank accession number NM_001368930.1, contained in NC_000011.10) code for transcription factors that play an important role in embryogenesis and both belong to the family of the homeobox genes. Specific hypermethylation of the promoter region of HOXA9 is known in connection with lung cancer (Wrangle et al., 2014, Functional identification of cancer-specific methylation of CDO1, HOXA9, and TAC1 for the diagnosis of lung cancer, Clin. Cancer Res. 20:1856-1864), hepatocellular carcinoma (Kuo et al., 2014, Frequent methylation of HOXA9 gene in tumor tissues and plasma samples from human hepatocellular carcinomas, Clin. Chem. Lab Med. 52:1235-1245), ovarian carcinoma (Singh and Sachan, 2017, HOXA9 and SOX1—a promising DNA methylation based diagnostic biomarker for epithelial ovarian cancer, Can. J. Biotech. 1:66), and head and neck tumors (Uchida et al., 2014, Investigation of HOXA9 promoter methylation as a biomarker to distinguish oral cancer patients at low risk of neck metastasis, BMC Cancer 14:353). In addition, HOXA9 may have potential as an epigenetic marker for predicting the course of disease in head and neck tumors (Hayashi et al., 2015, Correlation of gene methylation in surgical margin imprints with locoregional recurrence in head and neck squamous cell carcinoma, Cancer J. 121:1957-1965) and bladder cancer (Kitchen et al., 2015, Methylation of HOXA9 and ISL1 Predicts Patient Outcome in High-Grade Non-Invasive Bladder Cancer, PLoS ONE 10:e0137003). Tumor-specific hypermethylation of the promoter region of PAX6 is also described in various publications with a prognostic value for non-small cell lung cancer (Kiselev et al., 2018, Transcription factor PAX6 as a novel prognostic factor and putative tumour suppressor in non-small cell lung cancer, Scientific Reports 8:5059; Ooki et al., 2018, Epigenetically regulated PAX6 drives cancer cells toward a stem-like state via GLI-SOX2 signaling axis in lung adenocarcinoma, Oncogene 37:5967-5981).

In an embodiment, the one or more regions of genomic DNA associated with the specified genes ZNF773, ZNF671, ZIC1, HOXA9, and PAX6 whose methylation status is to be determined comprises those genomic DNA sequences within approximately 0.5, 1, 2, 3, 4, 5, 6, 7, 8, 9, 10, 15, 20, 25, 30, 35 or 40 kilobases upstream and/or downstream (5′ and/or 3′) of the sequence of at least one of the specified genes. In an embodiment of the present invention, the region of genomic DNA associated with ZNF773 can be between nucleotide 57,499,757 and nucleotide 57,500,375 of human chromosome 19. In an embodiment, the region of genomic DNA associated with ZNF671 can be between nucleotide 57,727,218 and nucleotide 57,727,600 of human chromosome 19. In an embodiment, the region of genomic DNA associated with ZIC1 can be between nucleotide 147,412,556 and nucleotide 147,412,790 of human chromosome 3. In an embodiment, the region of genomic DNA associated with HOXA9 can be between nucleotide 27,164,297 and nucleotide 27,166,843 of human chromosome 7. In an embodiment, the region of genomic DNA associated with PAX6 can be between nucleotide 31,798,513 and nucleotide 31,799,868 of human chromosome 11. The nucleotide numbers (positions) recited herein are from the Human Genome Assembly hg38 of December 2013.

Preferably, a portion of the foregoing sequences comprises a CG-rich region and/or a CpG island contained within the larger sequence. Thus, in an embodiment, the one or more regions of genomic DNA associated with the specified genes are one or more portions of the above-specified chromosomal sequences, in which the one or more portions comprise a CG-rich region and/or a CpG island.

In determining the methylation status of these regions of genomic DNA sequences or portions thereof, the methylation state of single, e.g., isolated, cytosines contained within these sequences can be determined, as well as the methylation state of cytosines in CG-rich regions and in CpG islands contained within these sequences. In a preferred embodiment, the methylation status of the one or more regions of genomic DNA associated with the specified genes is determined by measuring the methylation state of cytosines in one or more CpG islands contained within such genomic sequences.

As used herein, the terms “portion”, “fragment” and “part” are used interchangeably and refer to a fraction, in particular to a fraction of a larger nucleotide or amino acid sequence. Also encompassed within these terms is a molecule that comprises multiple discontinuous portions of a larger molecule, e.g., a nucleotide sequence which comprises one or more discontinuous portions of a different nucleotide sequence, such as a chromosomal sequence. In certain embodiments, a portion of a nucleotide sequence can be about 10, 20, 30, 40, 50, 100, 150, 200, 300, 400, 500, 600, 700, 800, 900, 1000, 1500, 2000, 2500, 3000, 3500, 4000, 4500, 5000, 6000, 7000, 8000, 9000, or about 10,000 nucleotides or more in length. In another embodiment, a portion of the chromosomal sequences of chromosomes 1, 3, 7, 9, 11 or 19 comprises at least one CpG island or a portion of the CpG island.

The tissue for which the risk for developing head and neck cancer is to be determined according to the present invention can be any tissue of the patient. Exemplary tissues include, but are not limited to throat, neck, mouth, tongue, nasal passages, esophagus, tonsils, and salivary tissues.

The terms “subject”, “individual”, “organism” or “patient” are used interchangeably and relate to vertebrates, preferably mammals. For example, mammals in the context of the present invention are humans, non-human primates, domesticated animals such as dogs, cats, sheep, cattle, goats, pigs, horses etc., laboratory animals such as mice, rats, rabbits, guinea pigs, etc. as well as animals in captivity such as animals of zoos. The term “animal” also includes humans. Preferably, the terms “subject”, “individual”, “organism” or “patient” refer to male and female mammals, in particular male and female humans. The subject can be of any age; however, it is preferred that the subject be an adult. In certain embodiments, the subject can be infected with a papillomavirus or can be free of infection of a papillomavirus.

The term “in vivo” relates to the situation in a subject.

As used herein, “biological sample” includes any biological sample obtained from a patient. Examples of such biological samples include saliva, blood, smears of cells, sputum, bronchial aspirate, urine, stool, bile, gastrointestinal secretions, lymph fluid, organ aspirates and tissue biopsies, including punch biopsies. Optionally, the biological sample can be obtained from a mucous membrane of the patient. Smears, saliva samples our mouth wash containing cells are preferred. The biological sample preferably can contain cells from the tissue for which the increased risk of developing a head and neck cancer is to be determined. Preferably, the biological sample contains DNA, e.g., genomic DNA or circulating cell-free DNA, such that the methylation status of the DNA or a portion thereof can be determined. The biological sample can be one that is obtained from the tissue for which the risk of developing a head and neck cancer is to be determined.

By “being at risk” or “has an increased risk” is meant a subject, i.e., a patient, that is identified as having a higher than normal chance of developing a disease, in particular a cancer of the head and neck, compared to the general population. In an embodiment, the increased risk means developing the cancer within 1 to 3 months of the sample being obtained. In an embodiment, the increased risk means developing the cancer within 3 to 6 months of the sample being obtained. In an embodiment, the increased risk means developing the cancer within 7 to 12 months of the sample being obtained. In an embodiment, the increased risk means developing the cancer within 13 to 24 months of the sample being obtained. In an embodiment, the increased risk means developing the cancer within 24 to 36 months of the sample being obtained. In an embodiment, the increased risk means developing the cancer 36 months, e.g., 42, 48, 52, 60 months or later, after the sample has been obtained.

In accordance with the present invention, the genomic DNA present in the sample can be processed in some manner in order to determine the methylation status of the relevant genomic DNA sequences. For example, the genomic DNA can be extracted from the biological sample and the methylation status of a particular region of the DNA can be determined using any method known to the skilled artisan, e.g., extraction with phenol/chloroform or by means of commercial kits and then determining the methylation using the sodium bisulfite or ammonium bisulfite method or by means of a commercial kit, such as the EZ-DNA Methylation-Gold™ kit, Zymo Research, Irvine, California. In another embodiment, the methylation status can be determined without the need for a preparatory step of DNA isolation from the sample. In another embodiment the methylation status can be determined on DNA in which other than bisulfite-based methods are employed.

The term “methylation status” in general refers to whether or not genomic DNA or a region thereof contains methylated nucleotide residues, in particular methylated cytosine residues, i.e., 5-methylcytosine. In an embodiment, a region of genomic DNA whose methylation status is to be determined is one that is rich in guanine and cytosine residues, and in particular is rich in CG-dinucleotides, i.e., the region contains one or more CpG islands. The methylation status can be determined by means of known methods, as discussed below. Methylation often occurs in promoter regions of genes, and thus, methods for the detection of the methylation status of relevant genes are usually concentrated in these regions. However, genes also can be methylated in regions other than the promoter region, since GC-rich areas such as those containing CpG islands can be located in other regions of the genes. The detection of the methylation status of such other regions of the genes is also encompassed within the present invention.

In the method according to the invention, the methylation status of preferably CG-rich regions, e.g., CpG islands, in the genomic DNA located between genes ZNF823 and ZNF833 or associated with the genes ZNF671, ZNF773, ZIC1, HOXA9, and/or PAX6 is determined. The term “methylation” is considered to be synonymous with the term “hypermethylation” as commonly known in molecular biology. It refers to the positive methylation status of the DNA, i.e., the presence of 5-methylcytosine in the DNA, preferably within a CpG island or other region rich in GC nucleotides.

As discussed above, the region of genomic DNA whose methylation status is to be determined can be located in an exon, in an intron, in the 5′ promoter/enhancer or any other region of one of the specified genes. As used herein, the term “is methylated” at least means that the DNA sequence contains 5-methylcytosine nucleotides. In one embodiment, the increased risk for developing neoplasia (abnormal and excessive growth of tissue) is determined by the presence of 5-methylcytosine nucleotides in the DNA sequence tested (for which the methylation status was determined). In an embodiment, the increased risk for developing neoplasia is determined by an increase in the amount of 5-methylcytosines (methylation) in the DNA sequence tested. The increase in the amount of methylation can be determined by comparing the amount of methylation in the biological sample to the amount of methylation determined in a control sample. In an embodiment, the increased risk for developing neoplasia is determined where the increase in methylation is at least 0.5%, 1%, 2%, 3%, 4%, 5%, 10%, 15%, 20%, 25%, 50%, 75%, 100%, 150%, 200% or more over the amount of methylation determined in the control sample. In an embodiment, the increased risk for developing neoplasia is determined where the increase in methylation is at least 1.5×, 2×, 3×, 4×, 5×, 6×, 7×, 8×, 9×, 10×, 20×, 30×, 40×, 50× or more over the amount of methylation determined in the control sample. In one embodiment, an increase in level of methylation is determined by whether or not the level of methylation in the biological sample exceeds a predetermined threshold level. A control sample can be a gene/sequence which is known to be either methylated or non-methylated, or can be the same sequence tested in the biological sample obtained from the patient but which same sequence was obtained from another patient, wherein which tissue in the other patient was determined not to develop neoplasia within a specified time period after the sample from the other patient was obtained. In certain embodiments, the specified time period can be at least 24 months, 30 months, 36 months, or 48 months or longer.

In an embodiment, the methylation status of the DNA is determined using nanopore sequencing, which interprets changes in ionic currents observed when single DNA molecules pass through a nanometer-size protein pore. In addition, nanopore sequencing is able to discriminate not only the nucleotides of a strand of DNA but also single base modifications, such as 5′-methylated cytosine. In view of these abilities, concurrent analysis of sequence identity and methylation of cytosines can be carried out, see, e.g., Euskirchen et al., 2017, Acta Neuropathol, epub prior to publication, DOI 10.1007/S00401-017-1743-5.

The methylation status of the DNA also can be determined after a preceding modification of non-methylated cytosine residues by the bisulfite method by means of what is called a methylation-specific PCR reaction (MSP) using suitable primer pairs. In the bisulfite method, non-methylated cytosine residues are converted into uracil using sodium bisulfite, whereas methylated cytosine residues (5-methylcytosine) are protected against this conversion. Since uracil has pairing properties differing from that of cytosine, i.e., it behaves like thymine pairing with adenosine, the conversion can be detected using specifically designed primers based on the fact that uracil binds thymine and cytosine does not bind thymine. MSP is an established technique known in the art for the detection of DNA methylation.

In the context of the present invention, the design of the PCR amplification primers used for the detection of the methylation status will depend on the location of the sequence within the genomic DNA sequence, e.g., associated with one or more of the specified genes whose methylation status is to be determined. For example, methylation-specific primers for such sequences can be designed to bind only to the bisulfite-modified sample DNA if certain cytosines were methylated within the primer binding sites. If these regions were not methylated before the bisulfite treatment, then the primers will not bind and no PCR reaction product is formed. Thus, in the context of the present invention, the presence of a PCR reaction indicates that the particular DNA region of the particular gene is methylated, and thus, that the patient has an increased risk for developing neoplasia in the tissue or already has a malignant disease.

A real-time PCR method (QMSP), which does not only permit a qualitative detection of the methylation but also a quantification of the methylated DNA regions, is particularly preferred. This MSP can be carried out in a fluorescence-based real-time method where the formation of the methylation-specific product is detected by the incorporation of a fluorescent dye, e.g., SYBR®-Green I or II (ThermoFisher Scientific, Waltham, MA) or EVA-Green® (Biotium, Inc., Fremont, CA). These methods are able to detect regions of methylated DNA in a large background of non-methylated DNA and are high-throughput methods particularly suitable for screening tissue samples (Shames et al., 2007, Cancer Lett, 251:187-198).

Alternatively, the production of PCR products by MSP can also be detected by a hybridization method after completion of the PCR, e.g., using strips or arrays with fixed probes to which the resulting PCR products bind and thus can be detected. Other techniques, include the use of methylation-sensitive DNA restriction enzymes to differentiate between methylated and non-methylated DNA or the high-throughput sequencing of DNA chemically treated with bisulfite for the detection of methylated DNA.

Another preferred method is a QMSP method based on the “MethyLight” technique, in which fluorescent probes are used for the respective regions of DNA to be tested for methylation. In a preferred example, a probe carries a fluorescent dye marker at the 5′-end and a quencher at the 3′-end, which probe binds to the PCR reaction product between the two specific amplification primers (see, e.g., Eads et al., 2000, Nucleic Acids Research 28:e32). Fluorescent dye is released as soon as the probe is decomposed after binding to the target sequence by the 5′-3′-exonuclease activity of DNA polymerase and the measured fluorescence reflects the amount of product formed. The number of reactions to be carried out can be correspondingly reduced for samples to be investigated in this method by using several oligonucleotides and probes (Shames et al., 2007, Cancer Lett 251:187-198). Suitable fluorescent dyes and quenchers are known in the art, e.g., fluorophore FAM™, HEX™, NED™, ROX™, Texas Red®, etc., and quenchers TAMRA™ or Black Hole Quencher®, available, for example, at ATDBio Ltd., Southhampton, UK or LGC Biosearch Technologies, Steinach, Germany.

In a particularly preferred embodiment, the determination of the methylation status can be carried out as a multiplex QPCR experiment. Such a multiplex experiment permits the analysis of the methylation status of several regions of genomic DNA in a sample, which are known to be correlated with an increased risk for developing neoplasia in a single assay. The multiplex method offers several advantages since the methylation status of the DNA region(s) set to be tested can be determined in one or two reactions per sample. This saves considerable time, sample material and material costs. In certain multiplex experiments, the methylation status of up to five genes can be determined. In addition, one further specific oligonucleotide each, the “probe”, is used for each gene. The probe carries at one end a fluorescent dye and is designed such that the fluorescent signal is not detected until the probe specifically binds to the PCR reaction products formed. The different probes will carry different fluorescent groups and therefore each fluorescent signal can be detected simultaneously. Such methods also can be carried out by means of “microarray” technology.

In a particularly preferred embodiment, the determination of the methylation status can be carried out as a digital PCR experiment. Such a digital PCR experiment permits in a single assay the qualitative and quantitative analysis of the methylation status of several regions of genomic DNA in a sample, which are known to be correlated with an increased risk for developing neoplasia.

Other methods known in the art for determining the methylation status can be used in accordance with the invention, e.g., methods based on a direct determination of the amount of specific product by fluorescence. For example, molecular beacon technology can also be used herein. Molecular beacons are oligonucleotides which are linked to both a reporter fluorophore and a quencher. The nucleotides at the 5 ‘-end of the probe are complementary to those at the 3’-end so as to form a secondary structure characteristic of molecular beacons. In this state, which is referred to as a hair-pin or loop structure, no fluorescence is detected due to the proximity of the fluorophore to the quencher. The distance between fluorophore and quencher is increased as a result of the binding of the loop structure to a complementary DNA sequence, which is generated during PCR, and thus fluorescence can be observed.

Another suitable technique includes the “scorpion” technology. Scorpion probes are complex oligonucleotides which combine the properties of real-time PCR probes and PCR primers in one (single-scorpion) or two molecules (bi-scorpion) Similar to the molecular beacons, they include a characteristic secondary structure having a self-complementary region whose ends were modified with a reporter fluorophore and a quencher. In addition, these probes can be used as PCR primers. During a PCR cycle, reporter fluorescence can be observed by the attachment of the loop structure to a complementary DNA sequence since binding increases the distance between the quencher and reporter fluorophore. For the detection of binding of different probes, the different probes can have different reporter fluorophores.

Furthermore, positive and/or negative control DNA, e.g., a non-methylated control region of DNA, can be co-amplified and used for controlling the PCR reaction and/or controlling for the presence and/or absence of methylation.

Moreover, it is known that the methylation of regions of genomic DNA is often connected with a transcription blockade of genes in proximity with these regions of (methylated) DNA such that the encoded protein of the methylated gene is not expressed. Thus, in an embodiment, an indirect determination of the methylation status of one or more of the specified regions of genomic DNA can be accomplished by determining the concentration of the encoded RNA and/or protein of one or more of the ZNF823, ZNF833, ZNF671, ZNF773, ZIC1, HOXA9, and/or PAX6 genes. The detection thereof can be done by any appropriate method known in the art, e.g., (for RNA) Northern blot analysis, RT-PCR, etc., and (for proteins) antibody-based methods or methods which are based on the determination of a biological activity of the expressed protein.

As an illustrative example, the method according to the present invention comprises the following steps: (a) isolating DNA according to a standard method from a biological sample obtained from a patient, e.g., a smear containing cells of the tissue for which the risk for developing a head and neck cancer is to be determined, e.g., using QIAamp DNA-Mini kit (QIAGEN, Hilden, Germany); (b) chemically converting the isolated DNA according to the bisulfite method, e.g., by means of a commercial kit such as the EZ-DNA Methylation-Gold™ kit, Zymo Research, Irvine, California, which converts non-methylated cytosines in the DNA sample to uracils by treatment with sodium or ammonium bisulfite and subsequent alkaline hydrolysis; (c) amplifying the relevant DNA by means of specific PCR primers for the methylated form of the DNA; and (d) detecting the presence of PCR products, which indicates that the DNA was methylated in the obtained sample.

The present invention is described in detail by the figures and examples below, which are used only for illustration purposes and are not meant to be limiting. Owing to the description and the examples, further embodiments which are likewise included in the invention are accessible to the skilled worker.

### FIGURES

FIG. 1 is a depiction of the genomic region from chromosome 19 between genes ZNF833 and ZNF823. The diagnostically relevant region rich in guanines and cytosines and especially CG dinucleotides is located between the two genes (CpG: 59, means region contains 59 CG dinucleotides; triple-framed). The region located at the 5′ end of ZNF833 (CpG: 20; double-framed) is not relevant for the present invention. Lower area: more detailed representation of the gene region, with the position of the diagnostic marker region in relation to the ZNF833 gene.

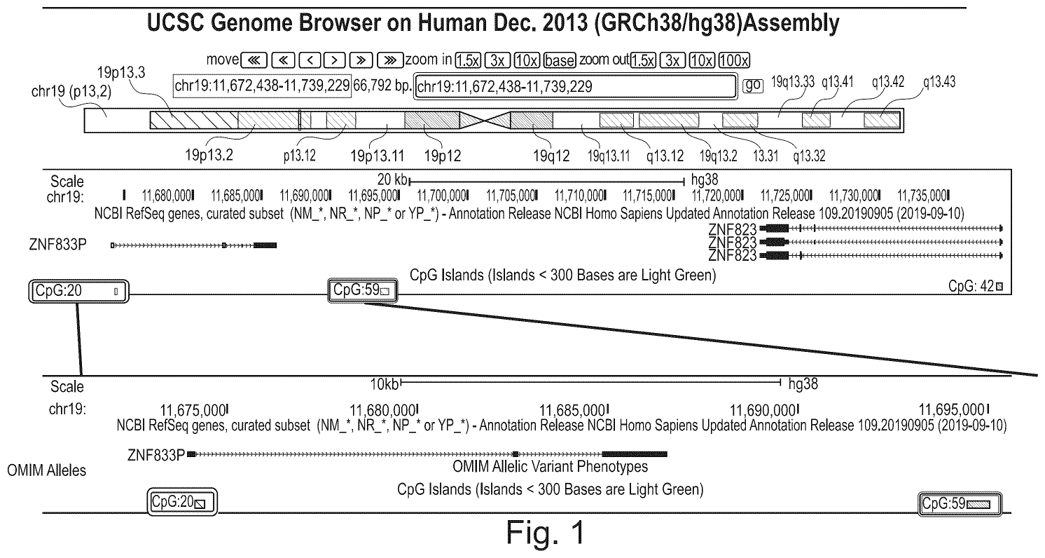

FIG. 2 is a depiction of the recognition rate of the markers ZNF671, ZNF773 and region between ZNF833 and ZNF823 for HPV-positive tonsil carcinoma (16x), HPV-negative tonsil carcinoma (28x), other head and neck tumors (19x), controls (56x).

FIG. 3 is a depiction of the detection rate of the markers HOXA9, PAX6 and ZIC1 for HPV-positive tonsil carcinoma (5x), HPV-negative tonsil carcinoma (11x), other head and neck tumors (12x), controls (24x).

### EXAMPLES

The techniques and methods used herein are described herein or carried out in a manner known per se and as described, for example, in Green, Sambrook, Molecular Cloning: A Laboratory Manual, 4th Edition (2012) Cold Spring Harbor Laboratory Press, Cold Spring Harbor, N.Y. All methods including the use of kits and reagents are carried out according to the manufacturers' information unless specifically indicated.

Diagnostic performance regarding the methylation status of the genomic region between the genes ZNF823 and ZNF833 on chromosome 19:

The determination of the association of the methylation status of the genomic DNA between genes ZNF823 and ZNF833 on chromosome 19, as well as with the marker regions ZNF773, ZNF671, ZIC1, HOXA9, and PAX6, and the increased risk for developing or having a cancer of the head and neck was as follows. Methylation status of a genomic DNA region (marker region) in the proximity of each of the respective marker genes was determined in tissues obtained from head and neck cancer patients and in healthy tissue from patients with non-malignant diseases as reference. Fresh-frozen tissue material was obtained and stored at −80° C. prior to testing. DNA was isolated from tissue using standard DNA isolation routines. As a control for the specificity of the marker regions, the methylation status of such regions also was determined in control tissue from patients without head and neck cancer.

The isolated DNA was subject to chemical conversion of all non-methylated cytosine residues, using either sodium bisulfite or ammonium bisulfite, followed by purification of the DNA according to standard methods. This chemical conversion is the prerequisite for the discrimination between methylated and non-methylated DNA sequences and thus for the detection of methylated DNA in a background of non-methylated DNA in the genomic regions of interest. This is of prime importance, since the biological sample analyzed usually comprises a mixture of cellular material and the aim of the method is to detect the few methylated DNA molecules originating from the subset of potentially precancerous and cancer cells of the tissue.

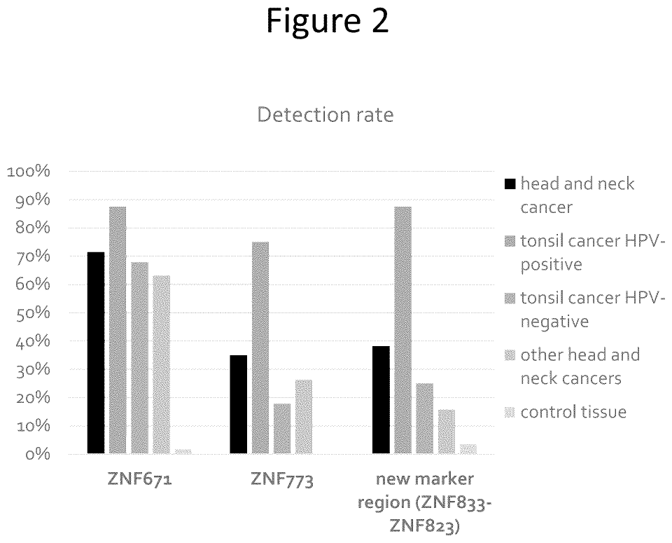

The oligonucleotide primers used for analytical PCR were designed to amplify the DNA regions of interest. Via methylation specific QPCR DNA, methylation the aforementioned markers regions were analyzed using the bisulfite-converted DNA from a cohort of 63 HNCs and 56 control tissues (FIG. 2) and 28 HNCs 24 control tissues for ZIC1, HOXA9, PAX6. In this example, the below-described primers only allow for the production of an amplification product where the DNA region of interest was methylated. The following PCR primers were used in the analytical PCR:

| | Localization | | |
|---|---|---|---|
| | of | | |
| | CpG region | | |
| | in the | | |
| | gene | | |
| | according | | |
| | to Human | | |
| | Genome | | |
| | Assembly | | |
| | Dec. 2013 | | |
| | (GRCh38/ | | |
| Gene | hg38) | Primer fwd | Primer rev |
| region | Chr. 19 | AAGGGACGGG | AACTAACCGTA |
| located | (len: 151) | ATAGGACGTTC | ACGAATCCCCG |
| between | sense | (SEQ ID | (SEQ ID |
| the genes | 11.694.599- | NO: 2) | NO: 3) |
| ZNF8233 | 11.694.749 | | |
| and | | | |
| ZNF8323 | | | |
| HOXA9 | Chr. 7 | GATTAATAGA | AAACCGACGAC |
| | (len: 202) | TTCGTTTGTT | GACTCCG |
| | sense | TCGTC | (SEQ ID |
| | 27166053- | (SEQ ID | NO : 5) |
| | 27166255 | NO: 4) | |
| PAX6 | Chr. 11 | ATTGGTAATTG | GCCTCCATAAA |
| | (len 243) | GTGTCGAATC | |
| | sense | (SEQ ID | ATCGAACG |
| | 31799442- | NO : 6) | (SEQ ID |
| | 31799685 | | NO: 7) |
| ZIC1 | Chr. 3 | TAGGGGATGCG | TACCCAAAACTA |
| | (len: 136) | GAGCGTC | CACACGATTTCG |
| | sense | (SEQ ID | (SEQ ID |
| | 147412090- | NO : 8) | NO: 9) |
| | 147412226 | | |
| ZNF671 | Chr. 19 | CGGAGGACGTAG | CTACGTCCCCGA |
| | (Len: 103) | TATTTATTCGC | TCGAAACG |
| | sense | (SEQ ID | (SEQ ID |
| | 57727371- | NO: 10) | NO: 11 |
| | 57727474 | | |
| ZNF773 | Chr19: | AGGTTTATTCGA | AACTACGAAAAC |
| | 57499833- | GAGTGCGAC | ACAACGCG |
| | 57499996 | (SEQ ID | (SEQ ID |
| | | NO: 12) | NO: 13) |

The results showed that the methylation status of the genomic region between the genes ZNF823 and ZNF833 surprisingly indicated a high sensitivity and specificity, especially in the detection of HPV-positive tonsil carcinoma (ToCa). The sensitivity for HPV-positive tonsil carcinoma is 88% for the marker region. The combined sensitivity for HPV-negative tonsil carcinomas and other head and neck tumors (HNSCC) is 38% for this marker region. The specificity of the marker region in control samples without malignancy is 96%. Thus, the detection of methylation of the marker region between the genes ZNF833 and ZNF823 in a biological sample indicates that a tumor in the head and neck region, with high probability an HPV-positive tumor of the tonsils, may be present in the person from whom the sample originates.

Altogether these results demonstrate that the determination of the methylation status of the genomic region between the genes ZNF823 and ZNF833 in a sample obtained from a patient provides a useful tool for early determination of an increased risk for developing a head and neck cancer and/or for diagnosing a head and neck cancer. It becomes clear from these results that the methylation status of this genomic region allows for an early diagnosis of developed head and neck cancer and maybe even the timely assessment of the risk to develop a cancer of the head and neck from, e.g., an already present precancerous stage.

Further marker regions of interest for the diagnosis of head and neck tumors:

Marker region in the area of the ZNF773 gene: A marker region in the area of the ZNF773 gene shows a similarly high sensitivity and specificity. The sensitivity for this region is 75% for HPV-positive ToCa, and 35% for other HNSCC. The specificity for this marker in control samples without malignancy is 100%. This would mean that a combination of this marker with the marker region between ZNF823 and ZNF833 enables sensitive and specific detection of HPV-positive tonsil carcinomas in a diagnostic setting.

Marker region in the area of the ZNF671 gene: The ZNF671 marker is characterized by very good detection specifically for cancer cells. It is already used for diagnosing cervical cancer. Surprisingly, the marker shows a good clinical sensitivity of 71% and a very good clinical specificity of 98% for differentiating between all head and neck tumors and control samples.

Since the use of several methylation markers in combination has proven advantageous in a diagnostic assay format, further methylation markers have been established. The diagnostic combination of the ZNF671 marker region with the marker region between ZNF823 and ZNF833 (sample positive=at least one positive marker from two) shows a good sensitivity of 78% and specificity of 95% for the detection of head and neck tumors (n=63) vs. Control tissues (n=56).

Marker regions in the area of the genes ZIC1, HOXA9 and PAX6:

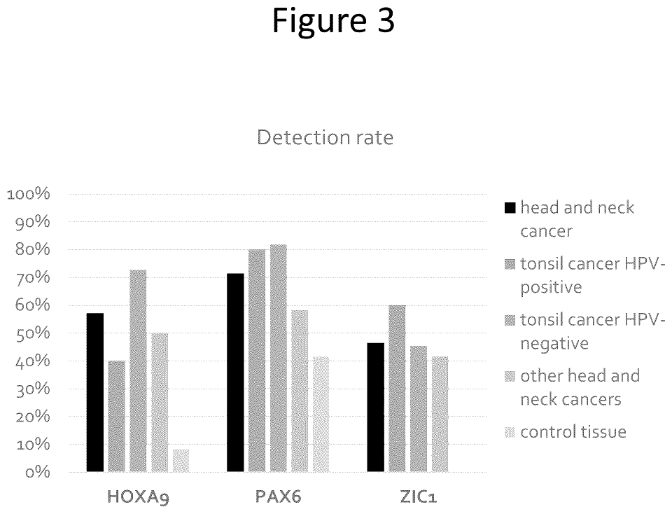

In addition to the aforementioned marker regions, three further biomarkers, DNA regions to be methylated in the areas of the genes ZIC1, HOXA9 and PAX6, mainly on swab samples from the oral cavity, were validated (FIG. 3). As a single marker, ZIC1 has a lower sensitivity (46%), but as a supplement to marker ZNF671 and the marker region between ZNF823 and ZNF833, it can increase sensitivity to 79% and specificity to 96% (tissue: 28 carcinomas, 24 Controls). ZIC1 has the potential to detect additional carcinoma samples that were not recognized by ZNF671.

The marker region between ZNF823 and ZNF833 and ZNF671 used in combination with PAX6 (in combination: sensitivity 89%, specificity 54%) and HOXA9 (in combination: sensitivity 75%, specificity 88%) also have a high potential as a head and neck tumor markers (tissue: 28 carcinomas, 24 controls).

We claim:

1. A method for determining the risk for developing a cancer of the head and neck in a human patient, comprising determining the methylation status of the genomic region between the gene ZNF823 and the gene ZNF833 on chromosome 19 in a biological sample obtained from the patient.

2. A method for determining the likelihood of the presence of a cancer of the head and neck in a human patient, comprising determining the methylation status of the genomic region between the gene ZNF823 and the gene ZNF833 on chromosome 19 in a biological sample obtained from the patient.

3. A method for diagnosing the presence of a cancer of the head and neck in a human patient, comprising determining the methylation status of the genomic region between the gene ZNF823 and the gene ZNF833 on chromosome 19 in a biological sample obtained from the patient.

4. The method according to any one of claims 1 to 3, wherein when the genomic region is methylated in the biological sample, the patient has an increased risk for developing a cancer of the head and neck, or the patient has an increased likelihood that a cancer of the head and neck is present, or the patient has a cancer of the head and neck.

5. The method according to any one of claims 1 to 4, wherein the level of methylation of the genomic region is 1%, 2%, 3%, 4%, 5%, 10%, 15%, 20%, 30%, 40%, 50%, 60%, 70%, 80%, 90%, 95%, or 100% of the maximum level of methylation.

6. The method according to any one of claims 1 to 5, wherein the genomic region is from nucleotide 11,686,870 to nucleotide 11,721,264 of human chromosome 19.

7. The method according to claim 6, wherein the genomic region is from nucleotide 11,694,447 to nucleotide 11,695,085 of human chromosome 19.

8. The method according to claim 7, wherein at least one of the CpG dinucleotides located between nucleotide 11,694,447 and nucleotide 11,695,085 of human chromosome 19 is methylated.

9. The method according to claim 7 or 8, wherein at least 0.5%, 1%, 2%, 3%, 4%, 5%, 10%, 20%, 30%, 40%, 50%, 60%, 70%, 80%, 90%, 95%, or 100% of the CpG dinucleotides located between nucleotide 11,694,447 and nucleotide 11,695,085 of human chromosome 19 are methylated.

10. The method according to any one of claims 1 to 9, wherein the methylation status is compared to the methylation status of a control sample.

11. The method according to any one of claims 1 to 10, wherein the cancer of the head and neck is cancer of the mouth, cancer of the nose, cancer of the nasopharynx, cancer of the throat, cancer of the hypopharynx, cancer of the larynx, cancer of the trachea, cancer of the esophagus, cancer of the tonsils, cancer of the sinuses, or cancer of the salivary glands.

12. The method according to any one of claims 1 to 11, wherein the biological sample contains cells of the head and/or neck.

13. The method according to any one of claims 1 to 12, wherein the biological sample is a tissue biopsy.

14. The method according to claim 13, wherein the tissue in the biological sample comprises cells of the throat, neck, mouth, nasal passages, esophagus, tonsils, and/or saliva glands.

15. The method according to any one of claims 1 to 12, wherein the biological sample is saliva, blood, sputum, bronchial aspirate, urine, stool, bile, gastrointestinal secretions, or lymph fluid.

16. The method according to any one of claims 1 to 15, wherein the method further comprises determining the methylation status of one or more regions of genomic DNA associated with a gene selected from the group consisting of ZNF773, ZNF671, ZIC1, HOXA9, and PAX6 in a different biological sample obtained from the patient or in the same biological sample obtained from the patient.

17. The method according to claim 16, wherein the region of genomic DNA associated with the gene is the promoter region of the gene.

18. The method according to claim 16, wherein the region of genomic DNA associated with ZNF773 is between nucleotide 57,499,757 and nucleotide 57,500,375 of human chromosome 19.

19. The method according to claim 16, wherein the region of genomic DNA associated with ZNF671 is between nucleotide 57,727,218 and nucleotide 57,727,600 of human chromosome 19.

20. The method according to claim 16, wherein the region of genomic DNA associated with ZIC1 is between nucleotide 147,412,556 and nucleotide 147,412,790 of human chromosome 3.

21. The method according to claim 16, wherein the region of genomic DNA associated with HOXA9 is between nucleotide 27,164,297 and nucleotide 27,166,843 of human chromosome 7.

22. The method according to claim 16, wherein the region of genomic DNA associated with PAX6 is between nucleotide 31,798,513 and nucleotide 31,799,868 of human chromosome 11.

23. The method according to any one of claims 1 to 22, wherein the patient is infected with papillomavirus.

24. The method according to any one of claims 1 to 22, wherein the patient is free of papillomavirus infection.

25. The method according to any one of claims 1 to 24, wherein the methylation status is determined by nanopore sequencing.

26. The method according to any one of claims 1 to 24, wherein the methylation status is determined by methylation-specific PCR (MSP), preferably wherein the MSP is a quantitative MSP (QMSP).

27. The method according to claim 26, wherein the QMSP is based on the use of fluorescent probes.

28. The method according to any one of claims 1 to 24, wherein the methylation status is determined by a methylation-sensitive restriction enzyme.

29. The method according to claim 28, wherein the methylation-sensitive enzyme cleaves methylated DNA.

30. The method according to claim 28, wherein the methylation-sensitive enzyme cleaves unmethylated DNA.

31. The method according to any one of claims 1 to 30, wherein after the patient has been determined to have an increased risk for developing a cancer of the head and neck, or the patient has an increased likelihood that a cancer of the head and neck is present, or the patient has a cancer of the head and neck, the method further comprises administering to the patient a medicament to treat the cancer or to prevent development of the cancer.

32. A method for selecting a patient to undergo more frequent screening for developing a cancer of the head and neck, comprising selecting a human patient in which the genomic region between the gene ZNF823 and the gene ZNF833 on chromosome 19 in a biological sample obtained from the patient is methylated.

33. The method according to claim 32, wherein the more frequent screening is histopathology-based screening.

34. The method according to claim 32 or 33, where the more frequent screening is every 12 months, preferably every 6 months, more preferably every 3 months.


SEQUENCE LISTING (13 sequences)

SEQ ID NO: 1 - DNA, 34395 bp, Homo sapiens
ttctagttggggacattatcaaggatgcagtttacccagagcgattaaccctagtggaggagctagattaggaaagtttctggggtcatggttaagtctaggttctacttggaatggatatctaaccagaaatctttcttgtatcattagtagagttgcccatttcaccaccgaagccatcaggacacaagagaagtctctaggttcccttgcctgcatggccctagacaaccacattgcagctatctccctactttacagggtagtgtctgtgctaacccttgcttctgcatctgggtaaaatacttccagtcagactgaattgggaacatataagatcttaaaaccagccaaatctctgaaagggacaccttcagaaaccctcctgtctagacttactgggttacatttccaatttccagatattttcagctggctcttcccctggtataggatttcttctccattctgccctacaagtctcaatgatcctatttaaaatatgcatttggctcctctttaaaattgttctatcttgtttcaacagatgcctccaagagacccccactaggttcttgctgacctaatacttgagattttaaactcactccagctggccactgaaaccaacttaacccaagagattttggctaaaatttaacaggttcctgagaacctcttataagtaaatggctatagttgcccaactggccactgccctgccattaggatccctgcatgggatggacccacagcaggtcactggtaccatgatagaacacacacaaaaacttatgaatgctgtctgtggacgggttagagtcagtccttcacactggctaactgggcctaagttttaaaaagagctaagccgccacttgttaactaggggtcacaagcactgtgagtttcctgaaaaccaacacctttttaactttgggggctcacctgagctagtcaatcagagctcagctgtatcaaccaatcagaactgagtttcaagtttcagatcttcatttgcataaaagcacttgactaggaacccagatgggaacttttgctataaaacctaagctttcccttgttctctggaacaaataatgtttaggttgcaaactccttatgccttttccccagaactatgtagaaataacgccttctaaaatgtgatcttgataccagggaactcctttttttttttttttttttttttttttttgagtcggagtcttgctctgttgcccaggctggagtgcagtggcgtgatctcagctcactgcaagctccgcctcccaagtggctgagagacaggtgcgcaccaccacgcccagctaattttttgtcatttagtagagatggggtttcaccctgttggccaagatggtctcaatcgacctgaccttgtgatctacgtgcctcggcctcccaaagtgctgggattacaggcgtgagccaccgcacctggctgataccagggaactcccagagcaaagcactaaatactttcattcctctaaggtctatgactctaatcagcagaaagtagttagagcagttatcattccttttccttcaagacttaggaatgaacataataggagggatttgtaaccacaacagattatgtggtacaacttctatataacaaagctgagttttcagttgccatggacccaacactcaaggtcacataatctgagcatgcccagatgaaccaaatgtgtaaccatgggtggaacctatatgcttggactgaggtgtggagattaaagtaaaaagtagatgctgggcacggtgactcatgcctgtaatcccagcactttgggaggctgaggtgggtggattgcctgagctcaagagttcaagaccacccagagcaacatggtaaaaccctgtctctgtgaaagatacaaaatattaaccaggtgtggtggcgcacacctgtagtcccagctacttgggaggctgaggcagaattgcttgagctccagaggtgaaggttgcagtgagcgagatcatgccactgcattccagcttgggctacagagtgaggctctgtctcaaaaaaaaaaaaaaaaaaaagataccacatggtggaatctaggatctaatcaggttgggccccggtatcatcccatggcaggatccaatcgagtcacattaacctggcatgagatcattgcaagatgcagtgagttcatgtcttattaccccacgcttataaaacttgccaaagcccccagctcagggagacatatttgagcatttcttcctttctccttgccagtcaaacttgtagtaaagcttttctttcttcaaaagctggtgccatgctattggcttgtgtgtacatctggaagtgagccaattcactacttgctaacataacaaagaatcagcaactgtgaagatgaaacaattaaaattatccagtctgggccaggcgcggtggctcacacctgtaatcccagcactttgggaggtccaggtgggtggatcacctgaggtcgggagttcgagaccagcctgaccaacatggagaaaccctgtctctactaaaaatacagaattagctgggtgtggtggtgcatgcctgtaatcccagttacttgggaggctgaggtagaagaatagcttgaaccaaggaggtggaggtagcagtgaaccgagattacgccattgcacaccagcctgggcaacgagagtgaaactccatctcaaaaaaaaaaaaaaaaaaaaagaaagaaaaaaattaccagtctgtggaaaagaaagaggaaagaaattgagagaatgtgttactagtatacctgccttgccagtcatgttaaagttcttcagagcgaaggaaactgatacaggttaggaaagcagatgtttcagaaataaaacttgttagaaaataattaaatgagggcacaaatatgattatcttttcttctccttcatagataaatgtttgttcaaatgaatatcaacaatgtattgggtggttatagcttctaacaggtcaaatgcgtgacagtgatattatatgggattggaaagaggaattcagaacactcttaacatattggcactaccccagacaccagtatagtgttatttgaaaatgaaagtccattagttgcaaatgtatactgcaaactatggaattcactaataaaatttaagaaacaagtagacttactacactaatagaagcaagcaaatggaatcatatagaacgctcaattaaaaggcagaaaaatagagcaagtagaaaaaaagcacaagtacaacaaacaatatatatgaacatattaatctatgtcaattaccactttgaatgtgtatttttttttttttttgagatggagtctcgctccatcgcccaggctggagtgcagtggtgccatctcggctcactgcaagctccgcctcccgggttcacgccattctcctgcctcagcctcccgagtagctgggactacaggtgcccaccaacacgcctggctaattttttatatttttagtagagacggggtttcaccatgctagccaggatggtctcaatctcctgacctcgtgattcatctcaggaagacaagatgggtggatcacttgagcccaggagttctcaagaccagctgagcaacatggagagaccctgtctctacaaaaaataaatacaaaaattagccaggcatggtagcacacgcctgtagtcccagatgaggtgggaggattgcttgagttcgggaggcacaggttgctgtgatcacaccactgcaccccagcctgggtgccagagaccctgtctcgaaaaataaataaataaataaataaaccaaaaaacctgatttatctgaatttaatatagccgttccatacttttgagtagggctagaatggcatatatttcaccatacctttctatttaaggtatctgtgtctttctgttgaaaatggttatcttttacacattgctgggccttgtttttaattctactctgccagtgcctgtcttttttactggtgtatatagataatacacattcaggccgggcgcggcggctcatgcctgtaatcccagcactttgggaggccgaggtgggtggatcaactgagatcaagagtttgagaccagcctaaccaacatggtgaaaccctgtctctactaaaaatacaaaaattagccgggtgtggtggcatgcacctgtagtcccagctactcaggaggctgaaacaggagaccatggaatattcaccaaggcagaccacatagcataaaacataccttaacaaatttaaaaaaatcaaattccataaaatacgaaatcaaaccgtattagcattaaactagagatcaatagcagaagcatagtttaaaagcctccagaaagtaaagatttaaaccacaaacttctaaactatgcaacagacagaattctccagagatacttaaatattccaaaaattgaaaaaaacttttagcaaaatttgtagaatacagcaaaggcagtgcttagagagctatttaaataattaaatgcaaactcttgaaagtaaagatctaaaaaacaatgctgtaaattccaccccaggaaactagagaaagaagagcacgataaatgtaaattaaggagaggaaaacaacgagagaagatattaatgaaattgcaaacaggaaatcaatggaaaacatcaacaaatccaaatcctggttttctgaaatgaataaaattgataaacacccatgctaactcacaaaagggtgggcatctctaagatttccatgaacattaaaagaaccataaaggaatattaactccatgcccatagatctgataaattacaagatgaggccgggcgcggggtggctcacgcctgtaatctcagcaccttgggaggccgaggcgggcggatcacgaggtcaggagatcaagaccatcctggctaacacagtgaaaccctgtctctactaaaaaaaatacaaaaaattatccgggcgtggtggctggcacctgtagtcccagctactcgggaggctgaggcaggagaatggcgtgaacctgggaggcgcagcttgcagtgagccaagatggcgccactgcactccagcctgggcgacagagcgagactccgtctcaaaaaaaaaaaaaaagtaaatctgagatgagttacagatgtcttcaaggtgacaaatccatggaggtgcagtgctgactgaaaacagacaagtctgactcaagtatcaggtcaggttctcctatcatctgagacataccccttcccatgctgctcactgaagtgctgagtgaccgccctcccaggagacactgcactgtgtgcttttgaggttcttgtaccacatcattggggggtgggttttcctttccttagggttagtttttacccttcactatgtggcaggccagagtctcactaacacaggcttccataacaactgtttcagcactgagtggttaaatattaaaagtcgagagagccgatgcctctaaacaaaggctggaatgtaacaaaagcccaacaagagttttgcccaggcctttcctgggccttgaagcatgacaagataacgaaggaattccttttttttttttttttttttttttttggagacggagtcttgctctgggactataagtgcatgccaccatgcccagctaattttcgtatttttagtagagatggggtttcaccatattggcaaggctggccttgaattcctgacctcaggtgatccacccaaagtgctggtattacaggcgtgagccaccgcgcctggcccagaaaacagcgttttgattgaatactatgacttaagaaatgtacacaaacactcgattctaatgggtatttttccctagaagggtatggattagcaattctgaaactattttacatgttagaatatatgttgttatgttatctacaatatatgttgtagataattagtgcctgtttctcactggagttgaaaaaagtaaaagaaataaagggacactgctagaatgaacccagtggtgctagaaatacagcaccagggaaaaagaatgtttaatgttgctgtgaactgaggagatgaaatcagagtcacaaaatgcagggaaagagcttaaaaaccaagggattttaagccctcggaggagttagttgagggaagagagtggtggattagaaaccctgattcccaaacctttggaagatgcagagggataccacttgccatttaggaatgtgaactcaaaagtatctgagacatgtttcaatttagaaagtttattggctgggcgtggtggcttacgcctgtaatcccagcactttgggaggcagaggcaggtggatcacctgaggcccagagttcgagaccagcctgaccaacagggagaaaccccgtctctactaaaaatacaaaattagccaggcgtggtggtgcatgcctgtaatcccagctactcgggaggctgaggcaggagaatcacttgaacccgggaggcagaggttgtaccgagccaagatggcaccactgcactctagcctgggcaagaagagtgaaactccatctcagaaaaaaaagtttattttgccaaggttaaggacacatccatgatacagcctcaggaggtcctgaccacatgtgtccaaggtggttggggtacagcttgcttttatattaatacattttggggagacataatacatcagtcaatacatgtaagaggtacattggttctacttggaaaggagggacaacacaaagtgggcgggggaggggtctggttcccaggtcacaggtggattttcccataaatggttgtggagaccaaagtttcataatgcagatgaagcctccaggtggcaggcttcagagagaatagattgtaaatgcttcttatcagacttaagttctgtgttgatgttaatactgataggcttttcctgggttccaaaagggaggcaaatataatggggcacgtccagctgatacagtttgactgtgtccccatctaaattgcatctggtattgtaatcctttcgatttaggctattgcgccacagagcctgcttgcaggaattgtaatcctatttgtaatccccatatgttgagggagggacctgctgggaggtgattggatcatgggggtggtttcccccatgctgttctcctgatacagtgttctcacgagatctggttactggataagtgtctgggctcctcccttctctctctccctctcctcccttctctctctccctcctgccaccccgtaagacgtgccttgcttccccttctgccatgattgtaagtttcctaaggccatgtggaacagtgagtcaattaaaaactttttttaaaaataaattacccagtctcaggtagtatctttccagcagtgtgagaacagacgaatacacagaccacccttccatgatggcctgaacaagtttttcaggttaattttgtagtgccttcgccgggaggagggatctgttcaaaatgactggggggcttagaattttatatttggtttaaaaaaggaataaaggaataaagaaggaactggggaccccggaccgtagctccttccacgcatgaaccccgcacacgagtcgggattccccccatgaccctcccgtggcccccgcacaatctggagagacgcggggctgcgggcgcggagctgcccagagaggactcctgcccgggcccgcagtcgccgcgaagggacgggacaggacgcccggggtcccggctgccagcccagccccaccctgcggccgaggggaccgagggccgagctccgccagcggtactccggtccacagagcccggagtcgctggctgggaggccggggacccgccacggccagttccaaccagcccctcctcccgtctcgggatccctggcccctcacgctcaccattttccgaattcctccgtgtcccgggggcctctctgcggctcccacgaccagtgcaggtccctgtgtgacagaggctgccgcagactctccagagtgcctctcagcgacagagacaggagcccagcgaagtggcgtgtagaagacgccgcgggctttttcaatctcgcaccctcttagctgaagtgcgcctgattgacagttcccacgaccccgccccacggccctgattggatagtgcgacagatcccgccccctgacgactgagttacagaagcgatctcacgctggggagtggagactgatgaacgagttcttgacacagcccttggcagggcgggcttcttccctgcgctgtgacctgacccctcccaggggacatctgcatttaagcaggatcccccatctgtactcagtgtgaccttcctgctttttcctcctagactggggatcacaagtgtgtgcagggaaacccagacacattgtccagtggagccattccctggcctcggagtaggaggagagccgaggccatacgggccacactgcaacaagaataagggcttaggctgggcgtgatagctcatagccgtaatcctaacactttgggaggccgaggccggtgcattgcttgagcccaggagttcgagaccatcctggacaacatagtgagagcctgtctctacaaaaaaatacaaaaattagcctggaatggtggcctgtacctgtagtcctagctactcggaaggctgaggtgggaggatcacttgagcctgggaggtcaaggctgcagtgagtcaagatcgtgccactgcactccagcctgggcgacagagtgagacaccatctcaaaaaaaaaaaaaaaaaaaaaaaaaaaataagggcccgatgtgttccagggatcaggaattttggataagtctgttggctgcatggccaagcctttctcccaccttatctctcaatctactgattttcagccaaggaagtaataacatgccaacaacggaataaaatcattaaatgtatggaaaaagtggaatcacatagtagcaatattttataaggaaatcgaaagacaagaaaagcaaacactcgaactagttgagataagtagcctgctgcacccacagttcacaaacctgcagtcagaaggtgatacacgttcccaagcttgtgagtcaaactctgatgaggcaggggcacttaaagatttaactctgggaactcaacgtggcctcctactccttctccagagtgtggaaatgctccttccttcaagaccttcattttctttctttctttttctttttttttttttttttgagacggagtctctctctgtcgcccaggctggagtgtagtggcgtgatcttggctcgcaaccttcgcctcccgggttcaagcgatcctcctaccttagccacccaaggagctgggactacaggcatgtgctaccacgcccagctaatttttttttttgagacacagtctcgctctgttgcccaggctggaatgcagtggcacgatctcggctcactgcaacctccgcctcccaggctcaagcaattctcctgcctcagcctcccgagtagctgggactacaggcgcacgccgccatgtccggctaattttttgtattttagtagagacgggatttcaccgtgttgcccaggctggtcgcgaactcctgagctcaggcaatttgcccaccttggcctccgaaagtgatgagattacaggtgtgagccactgcactcggcccactaatttttatatttttagagagacgaggttttaccatgttggccaggctggtcttgaattcctgacatcgggtgatccacccacctcagcctctcaaaatgttgggattacaggtgtgaaccaccgtgccggccaagaccttcctttttgccagcaggaaactctcatctaaaaatcagtttaatgcattgacctatataaactgtcaaattcataactccttttccagaaggcaatctaatgagtctttaagtctgaccttcacagattttcctcctatatacaaatgacagcctgcctcctgtgactctgtgcatgcccaaggcaaagcacttgaatgtactgagatcttcaatatttacaaaagaagatggtcagattaaaatattctgtctatgccctcaatgagcaggtagtataacaggttagagaggacaaaatcaaataaaatggacatggaacttacccatgatatcacagtctatcctggaggatttttgtggttgttttgttttgttttggagacagggtctctgtgtcaccaaggctggagggcagtgagtggtgtgatcccagctcactgcagcctcaagctcctgggttcaagccatcctcccacctcagcctcccaaggagctggaactgcaggcgtgcaccaccagcttggctaatttaaaaaaattttttgaggtagacacggcgtggcggggggatgggggagtctcaccatgttgcccaggctggtcttgaacccctgggctcaagcaaccctcccccctcagccttccacagtgctgggaatacaggtgtgagccaccgtgcctggcgatggtttctaaaaagcactttgcttcaaaggatttagagagccacaggtgggttcatctggggccagttagactgcttgaaaaggaaagctatttatgcaatctgcagctgcctaggaaatggccccaaagttcccagagttacagtctcaaaaaggacgcaaccccctagctgcatctacatgtgggtttcccgctttccagggcactgcagcttctcagaatccacagtccttagctgttctgagcagctggaggccatccgcaagggtaggcaggatgcccagctccattctcctccctttgtaggcttaagactggtgtcagcttaaagtcactgtcctcaggctctgctgcccactccaggtttttagggctccgaaaacaatgatcctcaaaacaggtctcagaagcagaagtttttctctgaccttctcttgccctcctgtctctcagtcccattctccaccaagggtagccatagaagctagaatccctcttccccaaggcaggtcatagaatccagaacccctattccccaagggcagccataaaatgcaaacagataactttccctgtgccttcctttggaaaaactggtcacaaagaatttatctgacctaccttatttgactgtaggtcataagactgtcatttaagagaggattctggccaggtgcggtggctcacacctgtaatcccagcactttgggaggccaagacaggtggatcacgaggtcaggagatcaagaccatctggccaacatggtgaaaccccgtctctactaaaatacaaaaaattagctgggcatggtggcacgcgcctgtagtcccagctactcgggaggctgaggcaggggaatggcttgaacccaggaagcggaggttgcagtgggccgagatcgcgccactgcactccagcctggagacagagtaagactccgtctcaaaaaaaaaaaaaaaagattctgtctcatacctagaaggaaggaatgctactcagagagaccaagaaaaatctagacagacaggccttctgggtttcctcactcagtctgttagcattcaatcataccttttttgttcaatcaggtttcttcatagctgttcacacattgttaaacccaagcacaaaaatgtacaacttaccctgtatctctgagtcttcattctgaaggctcctgtgtatatacattaaataaaatgtgtatgccttttctccaattaatcagtcttttgtgagttgatttttcagtgcaacatgagagggtaaaggagaactttccccatggcccctacaaggttattcacctgcctttcactgataagttaaatgagacaaagaccctcaatcccactgtttgaataagaagccagcagactcctgggttttctcctatgtttatatataaagtagaggaagaaaattctaagatattttatatgggttaaaaaaaaaaaaaagcctggagtccgggtgcggtggctcacgcctgtaattccagtactttgggaggctgaggcgggcggatcacaaggtcaggagatcaagaccatcctggctaacacggtgaaaccccgtctctactaaaaatacaaaaaattagccgggcgtggtggcgggcgcctgtagtcccagctacttgggagactgaggcaggagaatggcgtgaacctgggaggcgcagcttgctgagatcgcgccactgcactccagcctgggcgacagagtgagactctgtctcaaacaaaacaaaacaaaaaaaccctgaaaatggttggctatggtggttcaagcctgtcatcccagcactttgggaggcagatcacttgaggctaggagttcaagactagcctggccaacatagtgaaaccttatttctactaaaaatacaaaattagccgggcatggtggcccatgtctgtagtcccagctactcgggaggctgaggcatgaggattgcttgaacccaagaggcggaggttgcagtgagccaagattgtgccactacactccagcctgggtgacagagccagactctgtctcgaaaaaaagaaaaaaaattaaaaaccacctggatataattattcctttgagaaaacaaactgttattttcctcctaacagttgtataaaacaactaatcaggctgggcacggtggctcacgcctgtaatcccagcactttgggaggccaaggtgggcagatcacttgaggtcaggagttcaagaccagtctggtcaacatggtgaaaccccatctgtactaaaaatacaaaaattagcagggtgtggtcgtgcatgcctgtaatcccagctgcttgggaggctaaggcacgagaatcacttgaacctggggaggtggaggttgtagtgagccgagattgagccactgcaccccagcctgggtgaaagagcgaggctctgtcacaaaacaaaaaaaacaaaaacacacacacgcacacgcacacactaatcaaaagtgaactctcccctttttcctctgtttcagcagctccccaaccttcacagttcatggttgtcatcacgtgctctgtacctggtaatggacatgttcagtttacccatctgaaactctcaaccccactccatcaccactgacccatccaatcatttctgcagctaaattctaagcattgtataaatacctcttccctttcagcagtccagctggcccaggttgaatccacctgtggctctctcatttccctcaatttcaattattcatttgaaactatcttccaggatcgactgtgatatcttgtatgatgaccacattccctttatttgcttctgttcttcctaacccatgtcgccacctgttcattgagggcacagacagaatattctaagctgactatcacctgctgtaaattctggagtctcagcacaacctggagaaatccacagcccagagaggatgttaatgccatccttaccacaagagggagcttcttattttcacttctggaaaatggctaaaaacagaattttggagttttacatagcacaatatagatgatagaagtaaaggtaacaccattcaattttgtttcattttctttctttttttttttctttgagacagggtctcactctattgcccaggcaggagtgcattgtcacgatcacagctcactgcagcctcaacctcctgggctccagtgatgctcctgcctcaacctccagttagctgggaccacgggtgtgtaccaccatgcctggcttattttttaaaaagttatttgtagaggtaggggtctcactatgttgcccaggtggtcttgaactctgggctcaagtggtcctcttgcctcggcctcccaaagtgttaggattacaggtgtgagccactgtggccaacctggtttcatttttatgatttaacttttcctaggttaaaaaagcaaataagtccatgcacagtggctcacctctgtaatcctagcacttcgggaggtcgaggctggaggatcacttgagccaaggagtttgagaccagcctggagcctgggcaacctagtgacaccctgtctccaccaaagaaagaaagaaagagaaagagagagagggagagcaaaaaagtctgtgaatcttaactgaatttctctgaatatgagaagtgcaaggaaatatctctacaaacggaaagatgtaggagtaactgaggtggtacaaaggattctctctgagatggtgacatttactgtgagacctgaaataatatattagggcagtaaaacgttagggcagaggattcagggaagatggcagagtagcaagcataaaaaagctttctcaggccgggcgcagtggctcacgcttgtaatcccagcactttgggagactgaggcgggcagatcatgaggtcaggagtttgagaccagcctgaccagcatggtgaaactccgtctctactaaaaatacaaaatttagctgggcatggtggcctgcacctgtaatcccagctacttgggaggctgaggcaggagaatcgcttgaacccgggtgtcagaggttgcagtgagcagagattgcaccactgcactccagccgacagagcgagactccatctaaaaacaaaagcaaaaacaaaaacaaaaacaaaaccaagctgtctcagccgggtgtggtggctcacagctataatcccagcactttgggaggctgaggtgggcagatcacccggggtcgggaattcgagaccagcctgaccaatgtggagaaacctcatctctactaaaaatacaaaattagccgggtgcggtggtgcatgcctgtaatcccagctacttgggaggctgaggcaggagaatcgcttgaacccgggaggcagaggttgcggtgagccgagatcgcaccattgcactccagcctgggcaacaagagcgaaactctgtcttaaaaaaaaaaaaaaagctgtctccccacctagacaaccactggactgacagaatcatctgatgtagcaatgttataactgtggaggctattgaagctttgcaacttccataaaaaggattagacagtaaactatggctaattgtggtcattttatcacttagcacaatacaagctatcatccccacccttacccctgttgcaggcagctgcacatggagtgttttcctctaatttggcctcgggatctctctcagaagcgtggctataaactttagtgctggccaggcggggtagctcatgcctgtaatcccagcactttgggaggccgaggcgggcggatcacaaggtcaggagatcaagaccatcctggctaacctggtgaaacccagtctctactaaaaaaaatacaaaaaattagccaggcatggtggcgggcgcctgtagtcccagctactcaggaagctgaggcaggagaatggcgtgaacccgggaggcagagcttgcagtgagccgagattgcaccactgcacaccagcctgggccactgagtgagactctgtctcaaaaaaagcaaaaacaaaaacaaacaaaaagaactttagtgctgcattgacggggctccaggacacttggttgtggatatttacagtgtgactttcatggggtctaatatttaagtgttaacccataaccaaatgtccttcacaggaaacttgattatactggcagatgcccttatggttctacctctgccaagttaatctctaccaagatagccagatacccactctctacagagccctgacctggtaataagctagacataggtgtggcagtcaggtgagacgtgtgagacagcatcaggcaggccaatatatgcactgtgggagtttcagaaggagaagagacagagaaaggtcaggggagactatttggagaaataatagctgcaaatattccaaattagataaaagccatgaatgaatgctgtcatccaagaagctcaatgaactccaggtaggatgaatgcaaagagaaggacatgaagacatattatactcaaattgtcaaagccaaacataaagggagaaccttgaaggcagcaagaaagaagtgaatcatcacatataaatgatctttcataagatcatcagcagatttctcatcagaagcgctgaaggccagaggtagtgggctgatgtattcaaggtacttaaaaaaaaatggccacggtggcacatccctgcagtcccagctattccggagtctgaagtgggaggatcagttaaggcccaagagttcaaggccagcctaggtaacatggcaagactccatctcagaaacaaagacagccaagaatccaatatctggcaacattgtctttcaaaagtgaggaggaaattaaggcatgctcacatgaacaaaaactgaggtagttttgttctttttgtttttgccattagatgtaccctgtgagaaacgcaaaagccttctacaggttgaaatagaacactggacaatatctggaaggcgtaagcccaaataaagatctcagtagaaacaaatgcatggacaattataaatgctaatattattgtacaataattgtgaactccagtttttgtctttacataattgaagatacaaatccattaaaaattattagtctaaaagctagcattttagacttggcttttgaactctatattttgtttttttacacagtttaagagactaataaaaaagaaactaggttgggcgtggtggctcacgcctgcaatcccaacactttgggaggctgaggcaggcagatcacgaggtcaagagattgagtccatccttaccaacatggagaaatcccatctctactaaaaaaagaaatacaaaaattagttgggcgtggtggcgcacgcctgtaatctcagttacttgggaggctgaggcagtagaatcacttgaacccaggaggcggaggttgcagtgagccgagattgcaccactgcactgcagcctggagacagagagagactccgtctcaaaaaaaaaaaaaaagaaagaaagaaacgaatgcaccgaaaatatgtttgcgtttttgaatacataaagtataaagatgtaaatttatgacaccaaactgaaaggggtgtgaacaggcatgccatggagcattgtatttgcatgctatttatgttaaattggcataaatttagaatgttatggccttaggatggtaaacgcaatccccatggtaattaacagagcagtagtgtatacacaaaaggaaacaataaagaaatttaaatgtttcactacaaaaagcaattaaaagcaaaataatgcagaaatgcatgaaatgagaagaaaaaagctacaaggcatattgaaaacaaataggaaaatgacaaaaccaagtcctttcttatcaggaattactttatctatttattcattttgagacacagtgttgctctgtcgtccaggctggagtgcagtggtacgatctcggctcactgcaacctctgcctcctaggttcaagtgattctcatacctccaaagtagctgggattacaggcatgcaattctcctgcctcagcctcccaagtagctgggattacaggcatgcgtcaccacacccggctaatttatttctatttttagtagagacagggtttcactgtgttggccaggctggtctggaactcctggcctcaagggatctgctcacttcggcttccccaagtgctgggattacaggcgtgagccgcagcgcctggcaatgtgagttaattttagtgtataatgtgagaaaggttctcagttcatctttctgcaggtcaatatcccattttcccagcacattgttgaaaacccattccctatgaaagcatcttggcccatttgttgaaaatcaatgagccataaacaaagagtgtcttagtccatttgtgctgctatcgtggaataccacagactgggtaatttataaagaatagaaattgactttctcacagttcagaaggcttagaaatccaagaagacactggcatctggcttcagcattgctgcattctcacagggtggaagacaggagggcaagagagggcaaattgcattcatcaaaccctgtataatgccattaatctattcacgagggcagagtcctcatgatctaatcaccaaaaggcccctcctcccaatagtgttgcagtgggaattaagttctgacttataaattttagagagtacacaaatatttaaaccatagctaagggactctcaattctgccctgctggtctatatagctgacaatttgccagtaagtctggttactttactacccaccctgatgctgaagcatcccattccgtcccacatgatagtcatgcaggcatagaaatggttctaaagccgcaagagcatcccttagacaacctagatgtacttctattttaggatacatcctgtaaaagaaattttctgaagaacataataatgggtcatgccatggttgctccacatgaaacgctcggggcactgtcttcactcactgtaaaatcagcccaaggactgaactcatagctcttaggaaagcttgcacattggcaaacaagaaaactgacacagagagtaagcttccacacccacaatctcagctactcaggaggctgaggcaaggggattcttgaggccaggagtttgaggccagtttgaaccacacagccagattccacctcaaaaagaaaaaagaaaaagaaaacggctacaatttacacggataccagatacgctttgggagtctttcatgcagtggacacagtctcaaaacctggaggatttacaacttgttctggttacttattacctatcatctattaccaatggacatgtaatacatgcactattacaggctattcatctccctcccaaagtggctactgttcactgttcagtccatattaaggagacttatcctatgtctttcaggaatgatagggcttaaatactgttaaatagaaagacagacatgtacccctttatcctttctccccccaatttataagcctgccttttttttttttttttttttgaggtggggtcttgctctgtcacccaggctgcagggcagtggcatgatcacagctcagtgcagcctcaaccttccaggctcaagtgatcctccaacttcagcctccaaagtagctgagatcacagacatgtgccaccatgcccaaataatttacttttttatttttatttatttatttatttatttttatttttattttttttgagacagagtctcgctctgtcgcccaggctggagtgcagtggcgcgatctcggctcactacaagctccgcctcccgggttcacgccattctcctgcctcagcctccagagtagctgggactacaggcgcccaccaccacgcctggctaattgtttgtatttttagtagagatggggtttcaccgtgttagccagtatggtctcgatctcctgacctcgtgatctgcccaccttggcctcccaaagtgctgggattacaggcgtgagccaccgcggccggccatttttttattttttgtaaaggctgtctttaactctggttgatattactatcaggtaaatgtcccataatctgaaggagaccaatagatacaaaagagagacaaacaattgttggagagattatatatgcctcatgattagtaattatagtaacttgttaacaattgtcattgttaatgaaaatcagattataccaaacatactccttgtagcccctttctctctctcttttttttttttttcttttttttgagatagagtttcactcttattgcctgggctggagtgcaatggcacgatctcagctcattgcaacctccgcctcctgggttcaagcaattctcctgcctcagcctcccaagtagctgggattacaggcatgcgccaccacgcccggttaattttttgtattttttagtggagacagtgtttctccatgttggtcaggtggtctcaaaccccctacctcaggtgatccacccgcctcggcctcccaaagtgctaggattacaggtgtgagccaccatgcccgaccagcccctttcttttgatattttgctttgcactaatctcctaccagctgggacatttacagagataggaaatagttaagaaaattaaaaaaagaaaaaaaaagaaaaaaaagaaaagaaaactggctgtgccctggcatttacaaaatttctgacccaattatttctcacaacactacttacttgtaaatctcacacaacactgcttacttgtaaatctcacacaatttcattcccatgtatatattcaagagaattgagggcagggactcacacagacacatgtattctaattctatagcagccttattcacagtagtcaaaatgtgattgtggaaacccacatgtttatcaacatataaatggatgagaacagtgtgttttaactatacaatggaaaattattcaagcactaaacaagaatttctgatatgtgccaccacatggatggatcttggaaatattatgctaagtgaaattagccagatatagaagcacaaatactgtatgctttcacttatatgaggtgcctagcatagtcaaattcacacagagagaaaatagaatagagattatcaggacctgggggtgggagagaatagggaattatgtgtatgtatatgggatgctaagtagaaatagcaagtggtatggtaacatgtatagtataatcccatccagtatgtattttagtttgtaccagcattgaagaagggtcttccagatggacagtcccagtggttacacttgccataggcaacaatgcccctgggccacatcttggaagtctatttgaattacatttcaatgcaatttattcttatgaatactctaatgaaacagctcttatcaaggcacccagtgaccaaacacttgtcaaaatctacgtcaaccctgtttccttgtcttattcaaactctcagttacctgtgacagagttggccactctctcatctttagatactttcttctggaggcttccaggagaggacatccacctgttactcctcctgcctcaccagcctgcccagtctcttccaggctcttcctcctgctaacctcaggggttgccagcacttggactcctcttgagcactcaatttttaaatttattttttatttttatttttgagatggagtctcgctctgttgcccaggttgaagtgcagtggcactatctcggctcactgcaacctccacttcctgggttcaagcaattctcctgcctcaccctcccaagtagctgggactacaggcaaacgccaccaaagccggctaatttttgtatttttattagagatggggtttcaccatattggccaggctggtctcgaactcctgacctcatgatctgcctgcctcggcctcccaaagtgctgggattacaagcgtgagccactgcacctggctgagcagtcattttctgttataattcgctcctaactctgaaaccagttggcctgttctggacccggggcttcaaataacttccgcatgataatattctcaacattccatccagatctcccttgcaagctccagactcccaggtccagtggcctgctgaaaaatcttcccttgtttggctcaaatatgtcatggctgaacatgaaacgtaacccttgacttcccctcctgtcaactcccctgagccttgttcctctgagtaagaggcagcacccaggagctctgaacagtaggacacatccttgactcttctctcttttcacatcatcagaagttctacaggagctacctctgcctggttcactcccactcaggtttcaggttttaatttgagcatcagtccccacaccagggaagcctccactaacctctcagactcgtttgggaaccccctccatcatcatatgctctcagatccacacacccttctgtactgcactgaacactatcagcaccggtcctttatgtttatgtccagactccactaaatagtgggaatggtagggcctttctctaccatcccacccagctccaaaaacactgctcaggttcatatctagatttcgaaatacacaaccttctgttaacatccactaacactagccaagtccaggccagcacgacctatcttgcaactactactgtggttttcaaactggtcttccagctcccctctagcatctttttttcccaaccaagagaaggagtaaacttcttcaaacactaatgaaaactgctgctttgctcaaaatccagtaactgtctataacatggcgacaagatcccatgtgatctggcccctgcctacctattgaagcccatctattattaccttggcactctagctgtggtccaaggatcaacaacacagatgacacctggaagcaagttaggaaggcagactttctttcatttttgagacagagtttcgttcttgttgcccaagctggagtgcaatggcaagatctcggctcactgcaacctccacttcccgggttcaagtgattctcctgtctcagcctcccgagtagctgggattacaggcgtgcaccaccatgcccggctaattttttgtatttttagtagagattgggtttcaccatgttggccaggctggtctcgaactcctgacctcaggcaatccacctgcctcggcctcccaaagtgctgggattacaggcgtgagccactgcgcccggccaggaatgcagactttcatgccctactcttaatctgctgagtcagaagctgaatttgaagatccatatttcctgtgaaagtttgagaagtacatctcataagcttccccctccacctgattcagccctaagggtattgcatattcctcaccactgaggtatttgaatcaacaaaatgtcttaggtggcgtagacccaggagggctgcaaaatgcttgggcttgaactgggagaggacactagagagacagtcccaagaagaccatgcggccacggccgagcctgcctttccagcactgccatggcatgtcccactgctcctgaagtacaggaatggccactcatgggaactgaccgggagtgactgtgtgcgcagtcagcaacacactgaggctggagtgcttgtagcaggatgagccgcagacgaaacccctcagacatcacgttaaagaaggaagggctttattcagccaggagcatcggcaagactcacatcttaaaaaccgaactccccgagtgagcaattcctgtccctcttaagggctcacaactctaagggggtccgcatgagagggtcgtgattgattgagcaaacagggagtacgtgactaggtgctgcatgcactggcaattagaacagaacagaacaggacagggattttcacagtgcttttctcttcaatgtctgtaatctacagataacataaccgattaggtcaggggtagatctttaactaccaggcctagggtgaggcgccgggctgtctgcctgcggatttcatttctgccttttagtttttacttcttctttctttggaggcagaaatcgggcataagacaatatgaaggggtgtctcctcccttctgctcacacactgaggctggagtgctcgcaccctgctacccatgtgctcaccctgggattcggggaagcatgcacacaggcaagcaggaaaaacacgaggatcctggaaggggatgggcagaacgtgatggaggagagaggtttctgcttcctccaagggccaaaagtcactgtctgacatgtgaacgagatactctaacagagttcggggttggggtaggggcagatacccagtacctgctacaaaaggccagtagtaaagtcacaacaaataccaaaatgtcatgtttcaaaggctctttagataagcatcgattttaaaaccatataagaattaaacttgttgccaggcagcatgactcatgcctgcaatcacaacactttgggaaaccgaggcgggaagatggctcgaggccaggagttcaagaccagcctgagcaagatagcaagacctcatttctccaaaaaaaaaaaaaaaaaaaaaaaaaaattagctgagtttggtaccatgcaccagtagtcttaactacttgggagactgaggcaggaggatgtcatgagcccaggtgctcaaagctgctgtgagctatgattacaccactgcattccagcctggataacagaataagatgcgatctctaaaaattaaaaaaaaaagaattaaatctacttgtctattacacataaacttcaagcaactcaaggatccaagagaatacaaaattgaaagaaaaattttatcaactgactttaccaaaacaaacccaccacctattcaacttatgaaagctctttttaaaatcaacttttattttagattcagggagtacacatgcaggtttgttacttgggtattttgcatgatgctgaggtttaaagtatgattgattccatcacccaggtagcgagcatagttcccaacaggtggtttttcaacctttcacctccctccctcccccgtctactagtccccagtatgtattgttcccatctttatgtccatgtttactcaatgtttaactcccccctttttttttttttgagatggagtctcgccctgtcacccaggctggagtgcaatggcacgatcttggctcactgcaacctccacttcctgggttcaaatgattctcctgcctcagcctcccgagtagcttggattacaggcacctgccaccacgcccagctaatttttgtatttttagtagagacagggtttcaccatgttggccaggttggtcttgaactcctgacctcgtgatccacctgcttcggcctcccaaagtgctgggattacaggcgtgagccactgcacccagctgtttaactcccatttctaagtgagaatatgtggtatttggctttctgttcctgtgttaatttgtttagggtaacagcctccagctgcatccatgttgcatcaaacgacatgattttattctttttttttttttttttgagacagagtcttgctctgttgcccaggctggagggcagtggcacaatctcggctcactgtaacttccaccaggttcaagcaattctcctgcctcagcttctcgagtagctgggaccacaggcatgcaccaccacagtcggctaatttttgtatttttactagagacggggtttcaccatgttggccaggctagtctcgaactcctgacctcaggtgatccacgcctcccaaaatgctgggattataggtgtgagccattgcccctggctgatttttttttttttttatggttgtgatgaacctttgtgtaaagaaaatttacaaagatatctcacattaattttttaatataagacttaggaatccagagtgaagatctattttgtcaatgagccactgataaagacaatatttatgtattttataacatttctttgatgtggcaaattttctggaacattatacatgagtaaatttggttaagaatttcctgaattacttacgtacatttacctccagtcgtgtgaggatgaggagccttcaagccaacaaagttgtagacctatatccatggaagtgacgagcaggtcatttcatcaggaaactctcacacttgtcagcagatagatgctgcacatggaattagttctattcctcagaaatttatatatatttgtaagaaatgtgatttaaattcaatactatatagtttatgttattgtgtacacagcatatgtttactccataagcaggaccttgactccattatcaggtttaatacttttccaggtgttttgtttttagtttcctgggcaaatagtgctatcatctccaatcacgatacttgcttcttccatctggttcttcatagtgatcttttttttttgtttttgtttggagactgagtctcgcactgtcacccaggctggagtgcagtggtgcaatctcagctcactgcaaactctgcctcccgggttcaagcgattctcccacttcagcctcccaagtagctgggattataagcggccgccaccatgcctggctaatttctgtatttttaggagatatggggttgcaccatgttggccaggctggtcttgaactcctgacctcaggtgatctgcccgccccggcctgccaacataatgatcattttttgacagtaagtactatagactaccgtaataataagtggggttaaacagacatatttgctttcattagattctgttcctcctttcactgggaagcctctcacctaccggttcctcatcgtgctgtgctctgaaacataccttaatgaagaatctgatatccaaaaaattcatcttcaaaatggaagacaaaagaaaaactatcccaggcttaaaatatatatattgagagaatgtgttcctactacaccagacctacaagaaagatcaaaggagggtcttcaatttgaaattaaaaaaagtcaaaatccacagggaaaaacaaagagcaccattagagattatgtaagtgggaacgacaaatggtatgtgtatttcttcatctttcttctcgttatagatttaaaaggcaactgtgtaaaataataagtagtcaatggattgttgggcctacaacatagaagaatgtaacatgtgcattatatttacaaatgaaaccataaaaaagtgagctaactaaatgactaccaaagcaaagtagaaagaatgtattgttggatgtgtaaacctaaggattgttacacatgcaacaaaataccccagaaaggcaaaaaatataatagggctttagaggataaatatgtttgtatatcactggaatttatccatagatcactggaaaattgaccctgattgggataagttatttacatgataaacacttgagtaaacactacagaacctcaagtcatcctacattgtaaagacgtattggatgaaagcagaatttactgtccttcctaaattaaaaatggacactacatacattctataacattatgaggacaagatttccctcattcatccaacttggaagtttccatgttatattacatatttccccttcaacattcactctacaattagaatcagaaaattgtttcttgtgcaggtcgtgatctctctatatccatttcttgaacttctagcccaggtacagaattacatttcaacgatcccttattttgcattatatgttctcaaaaaattttttttttttgagacagagtctcgctctgtcacccaggctggagtgcagtggcgcaatctcggctcactgcaagctccgccttctgggatcacgccattctcctgcctcagcctcccgagtagctgggactacaggcgcctgccaccacgcccagctaattttttgtatttttagtagagacggggtttcaccgtgttcgccagggtggtcttgatctcctgaccttgtgatccacccccctcggcctcccaaagtgctgggattacaggcgtgagccaccaggcccggcctccctgactttttttatggtgcaaaaatacacataaaataaaatttaccctctttttttttttttttgagaaggggtctcactctgtcaccaagttggagtgcagtggcacgatctctgttcactgcaatctctgcctcctgggttcaagcaattctcctgcctcagcctccccagtagctgggactataggcgtgcgccaccacacccagctaatttttgtatttttagtatagatggggtttcaccatgttggccaggatggtctcgatctcctgaccttgtgatccccctgcctcggcctcccaaaagttctgagattacaggcatgagccactgtgcctggacaaaatttaccctcttaaccctattcatgagggccctaccctcatggtctcatttcatcctaatgtcctcccagagatcccatctcctcattccatacattagagagtagtctttcaacgtatgaatttcaggggacaaataaactcaataatattgtaatctccaccccaatgtatttcctcacatgcaaaatacatttattccatcccaacaccatcaaaagtcttgactcattctatcattaactctgaagcctaaaatcaaagtctcatttaaatagcatctgaatcagataagggtgagagtcaatgtaacattcatgctgagccaacattcttctccagatgtaaaatcaaatcagttgtgtacttttaaattataatggtggaacagttataagataaacaatgctattccgaaggggagccatataaggggtggaagagtgacagttcccaagcaagtccaaaacctggaaaggtaaacttcatgagatttttttttttttttttgagatggagtttcgctcttgttgccaaggctagagtgtaatggcgcgatctcagctcactgcaacctctgcctgccgggttcaagcgattctcctgcctcagacccctgagtagcttggattacagccatgtgccaccacgcctggctaattttgtatttttagtagagatggggtttctccatgttagtcaggctggtctcgaactcccaacctcaggtaatccgcccgcctcggcctcccaaagtgctgggattacaagtgtgagccactgcgcccggccaggatcttaaggctaaataataatcccttttcattcaatactctgccctcaaggaattgtgacacagcaattgtgtcccagcatctctgttagacatggatcaaacccaaatagcgtttgatggcactgcatccacacttctgggtttagctcatcttgtttgtggaaactccaaaatgtcccctactgttgatgctcaggaggcagccttcatgacctctaaacagctactgggatccttcttctcttgtctctaagaataccatgcattcagctctatggatggttcctgtaaaatcatacaggtccaacaactttcattttttcctgtgaccttcactttcagttcaaaggggctattttactgctgaggtggctaagtccatggttaacaagaataccactcttcctatcaaaaggtcttggacacacccttggtgctgtcttcccagcatgccttttcatctttgcgataattgttttctaaacattttcatttcaatttctcaatttcttcttgcttaataattccatctccaacatttttctctactcacattttatttatttatttattgagacagaatctcactctgtcgcccaggctggagtgcactgttgcgatctcggctcactgcaacctctgccacttgagttcaagcgattctcctgccttagcctcccgagcagctgggattacaggcatgtgacaccacatctggctaatttttgtatttttagcagagacagggttttaccatgttggtcaggctggactcgaactcctaacctcaagtggtctgcctgcctcagcctcccaaagtgctgggattacaggtgtgagccaccgcgcccggcctctcctcacatttagtatatgcagtcaagaggatccaagttgttttttttttcctttttttttttttttgagacagagtctggctttgtcacccaggctggagtgcagtggcgcaatctcggctcactgcaagctccacctcctgggttctcaccattctcctgcctcagcctcccgagtagctgggactacaggcgcccgccaccatgcccggctattttttttgtatttttagtagagacggggtttcactgtgttagccaggatggtctcaatctcctgacctcgtgatccacctgcctcggcctcccaaagtgctgggattacaggcgtgaacccaccacgcccggcccaggttgtttttttcaacacttctaagaaatattgtcagctcaatatcaagtttcatcacttcctagttctagtttccccaaaacagtagcaccggagtacaatgcaccaagttctttgccactttctgcaacggttcatcttttctctattatgcagtaatattgtcctcaattctgagacctcattagtattgcctttattgtccatatatccaccaaaattttgtacatgattttttatgtgccctagtaagatggaagctttctctattcctctttttattttatttttttagagaaggggtcttgccacactgcctaggctggtcttgaactgctaggctcaagcgatcctcctgcctcaccctcccaaagttctggcattataggcatgaaccaccataccccgcctgcctgtcctcttttgtgagctcttaccagcgtccaaagacaaaattacaacaaatttaggtactggcagatttagtgtctgatgagggcctgttttctgattcacagacagctgtcttttttgtttattttttgaggcagagtctcactctgtcacccaggctgcagtgcagtggtgtgatcacggctcactgcagcctcggcttccctgggcttgggtgatcctcccacctcagcctcttaagtagctggaactacaggcgtgtgccaccatgcccagctcatttttgtattttttgtagaaatggggctttatcatgttgcccaggctggtcttgaactcctgggctcaagtgatctgcccacctcagcctcccagagtgctgggattacaggcatgaaccaccaggcctagcctcatagataactatcttaatgtatcctccaaggcagagagcaaaaaagcaagctgctgggcggactcacaataggtaaccaatttcattattgaggctccactctcatggtctaatccaatctgtattatattccaaagaagacgttccctaatctgtcacgttagggaaatggattagaatgagtggattttatgaagacatttattccctaacaggtggtatgctttttgttgttgactttttagagttctctaagcagtctgcatattaatttcttgtcagatacatgatttggaaatactttctctcattctatgggttgtctattttgatgccgaaagtttaaaaattccatatatttccatttattctcttatgggtcatgcctttggtgtcatatctaggaaatcattggaaaatccaggttcataaagcttttgccttatactttttttttttttttttgagacggagtctcactctgtcaccaggctggagtgcagtggcgcgatctcagctcactgcaacctctgcctcctgggttcaagtgattgtcctgcctcagcctcctgagtagtgggattacaggcatgagctaccatgcccggctaattttgtatttttaatggagacggggtttctccatgttggtcaggctggtttcaaactcttgaactcaggtgatctgccagccttggcctcccaaagtgctgggattacaggcgtgagccaccgtgcctggacagggaagggtacattttcattctgttgcatatggatgtgcatttttccccagcatcattctatggaaatactgtcctatcctccattgtatcgtcatagtacgcttgtcaaaaattgttggactcactatgcaaacgtgtatttctgtgctgtctattttgtcacaatggtctatttctttgtcagtatgctgatattaaatttttttattagtataaatttgctctaagttttgaaatcaggaagtgtcaatctaccaactttgctcttgtttttcaattttattttttatttatttatttttttgagacggagtctcgctctgtcgcccaggttggaatgcagtggcgcgatcttggctcactgcaagctccacctcccaggttcaagccattctcctggctcagcctcccgcccgagtagctgggactacaggtgcccgccactgtgcccggctaattttttgtatttttagtagagacggggtttcaccgtgttagccaggatggtctggatctcctgaactcgtgatccgcccgcctcggcctcccaaagtgctgggattacaggcgtgagccactgcacccgacgtttttcaattcttgttttgtatattcagagtcccttaagatgccatatgtaaaacaatggaaaaatgggtcctaaaaactaatcaagtggtcaaagctatccctaaaaggctagcagggagcaaaaagtgctatttctcaatgagaaaataaaaatcttagagagatgttttgaaaaatgccatgtcacttttgaaagtgggccacatggtaagaacaaactaagcattcacacactaaagcagaaagaagctgaaattcttggaagtcttagagctgcctctacaatgggaaaaatgacccctctgcttcatgataaactgagtaaaaagactgagaaagcattaagtatgtggctagaggacacatcgctgaacaacatctcttttgataataaaataaagtgggaaaaaacagcctctataaataccactgtgagaaggctgaggagagcaaggaaaaggagtttcaggccagtattgtctggctgggctagctacataaagcgccagagcctcaaaaatttaaagaccacaggagaattgacactggcttatgccaaggcaatattagcattcccagcagagctcacaaaactcataaaaaggaagtctaccttccagagcaagtcctccattccaatgaattgggcaagccctagaaaaagaaagcggtccagtggtacctacatccatgactgccaaggaggccctggggctcagggcctggaaagatcacctactctggtgcagtgtggcaatgtggcaggtcacaagaacaagccaggccttggctactgagcaaacaatccctaggtcctttagaaccaaagcaaaacttgcctgcctcctttctggcaaaaatataagaaggcttgggtgatgatcatcttgtccttggaatggttctaccaaggcttcattcctgatgtgaagaaatatcttgaagagaaggggctgccactcaaggtccttctcataattgacaatgttcctggccatctccaatctctctactttgccacaaagaatgtggaagtgatgtccctgctgcctaacactacatcacagtgaaagctactcaatcaaggcataatcaagtagatcctgaccttcgggaggattcatgccatcctgatgctaactctgactgtaccatagtggatttatgaaagcacttcacaattgcacatgcaactgtccttattgcagaggctatagatgtcccagagctcaagaaactcaaagcatgctggaagccattatggagtgagggagtcaataattttaggggcttccccactattgatgcagtagttaggaatatcttaaatactgcaaggggagttggtaagaaaggctatctctgacaggattgaagacgatgttgagtaacacacagaagggcataaattcttacaacaaggaactcaaagatctgctcaaatgctctatagattgatgatgataatgatgacttaaaaaaggtagtgccacagatttggacacttgaaaaatttgcagtagttttttttattttttttgagacggagtctcgctctgtctcccaggttggagtgcagtggcgccatctcggctcactgcaagctccgcctcctgggttcacaccattctcctgcctcagcctcctgagtagctgggactacaggcgcctgccaccatgcccagctatttttttgtatttttagtagagacagggtttcactgtgttagccaggatggtctcaatctcctgacctcgtgatccacctgtctcagcctcccaaagtgctgggaatataggtgtgagccacctcgcctgacctgcagcagtttttcaataggcaaaactgttaagaaagttcatcatcctcaagtaagatccttcgatggaacaaagcctccatgtcacccatggaataacatgccaacaaacagtgcaagatttttttgatgactcaaaaaaaaagaaaaaaagaaaaactagcttcctacacttctcactaagcacctgcaaatataaacacagagccttccatggttaaggatctgcagccctgaagctgaacagatcttgacattagtattatttggcctcctccaacaagtcaacatctgcatcagaacctagttctttgaacactgatgatcatcctgatattctgtcaaggttcaagttagcctgatgtctcctgcttgtctataatgctacaacacatgacaatgttaggcaccatccagcaatcatcatcatcatcattatacttgggttatcaagagagaagcaggattttgataagtgttgacatgctcattcaggtcacaaaacaaaagcaatctg

SEQ ID NO: 2 - DNA, 21 bp, Artificial Sequence
  fwd primer
aagggacgggataggacgttc

SEQ ID NO: 3 - DNA, 22 bp, Artificial Sequence
  primer rev
aactaaccgtaacgaatccccg

SEQ ID NO: 4 - DNA, 25 bp, Artificial Sequence
  primer fwd
gattaatagattcgtttgtttcgtc

SEQ ID NO: 5 - DNA, 18 bp, Artificial Sequence
  primer rev
aaaccgacgacgactccg

SEQ ID NO: 6 - DNA, 21 bp, Artificial Sequence
  primer fwd
attggtaattggtgtcgaatc

SEQ ID NO: 7 - DNA, 19 bp, Artificial Sequence
  primer rev
gcctccataaaatcgaacg

SEQ ID NO: 8 - DNA, 18 bp, Artificial Sequence
  primer fwd
taggggatgcggagcgtc

SEQ ID NO: 9 - DNA, 24 bp, Artificial Sequence
  primer rev
tacccaaaactacacacgatttcg

SEQ ID NO: 10 - DNA, 23 bp, Artificial Sequence
  primer fwd
cggaggacgtagtatttattcgc

SEQ ID NO: 11 - DNA, 20 bp, Artificial Sequence
  primer rev
ctacgtccccgatcgaaacg

SEQ ID NO: 12 - DNA, 21 bp, Artificial Sequence
  primer fwd
aggtttattcgagagtgcgac

SEQ ID NO: 13 - DNA, 20 bp, Artificial Sequence
  primer rev
aactacgaaaacacaacgcg